(19) United States
(12) Patent Application Publication
(10) Pub. No.: US 2014/0187448 A1
(43) Pub. Date: Jul. 3, 2014

(54) Radio Frequency Identifiers for Use in Biological Science

(71) Applicant: LIFE TECHNOLOGIES CORPORATION, Carlsbad, CA (US)

(72) Inventors: Matthew O'Banion, Vista, CA (US); Traci Libby, Carlsbad, CA (US); Charles Piazza, Guilford, CT (US); Emanuel J. Vacchiano, Burr Ridge, IL (US)

(73) Assignee: LIFE TECHNOLOGIES CORPORATION, Carlsbad, CA (US)

(21) Appl. No.: 14/088,275

(22) Filed: Nov. 22, 2013

Related U.S. Application Data
(63) Continuation of application No. 13/037,801, filed on Mar. 1, 2011, now abandoned.
Application No. 13/037,801 is a continuation of application No. 12/181,155, filed on Jul. 28, 2008, now abandoned.
Application No. 12/181,155 is a continuation of application No. 11/269,509, filed on Nov. 7, 2005, now abandoned.
(60) Provisional application No. 60/625,441, filed on Nov. 5, 2004.

(51) Int. Cl. G01N 35/00; C12Q 1/68

(52) U.S. Cl. CPC G01N 35/00732; C12Q 1/68
USPC 506/39; 422/69; 204/600

(57) ABSTRACT

Provided herein are biological research methods, kits, and products that utilize radio frequency identifier technology.

### CROSS REFERENCE TO RELATED APPLICATION

This application is a continuation of U.S. application Ser. No. 12/181,155 filed Jul. 28, 2008 (pending), which is a continuation of U.S. application Ser. No. 11/269,509 filed Nov. 7, 2005 (abandoned), which claims benefit of priority under 35 U.S.C. 119(e) to U.S. Provisional application 60/625,441 filed Nov. 5, 2004 (expired), entitled “Compositions and Methods for Using Radio Frequency Identifiers in Biological Sciences” all of which are herein incorporated by reference in their entirety.

### BACKGROUND OF THE INVENTION

1. Field of the Invention

The invention relates generally to labeling biological research reagents and more specifically to using radio frequency identifier technology in biological research.

2. Background

The quality of biological research reagents is critical to the outcome of biological and biochemical experiments, as well as procedures such as diagnostic procedures, environmental sampling, and forensic testing that use biological research reagents. In many cases, biological research reagents include compounds that can function less effectively over time, for example, because of oxidation or other chemical reactions that can cause reduced or altered function of the reagent, side reactions during the experiment, test, or procedure, or otherwise lead to nonoptimal results. Although dating of research reagents by printed labels, embossed labels, stamps, or bar codes can prevent the user from employing a research reagent that is older than its recommended shelf life or expiration date, these systems all require a user or person maintaining the reagent stocks to manually check each individual reagent. This is impractical and time consuming, especially in cases where many vials, tubes, packets, cartons or other containers of reagents are stocked, as is often the case with biological research reagents. Biological research reagents frequently need to be stored under special conditions, for example, at 4 degrees C., −20 degrees C., −80 degrees C., in vacuum packs, or in dessicators, to enhance their shelf life. This makes taking inventory and taking account of past due date labels even more inconvenient.

Errors due to mislabeling or mishandling of biological research products can have serious consequences, as the result can be the loss or waste of biological samples that can be difficult, costly, or time-consuming to replace; lost time to reproduce experiments or tests; or errors in diagnosis or interpreting results of experiments.

In addition, biological research reagents are often used in procedures that can be performed under a range of conditions. Often the optimal conditions for a given sample or desired test must be determined by a researcher or operator by consulting a manual or other reference guide. This aspect of experimental or procedural set-up can be time-consuming and lead to errors, for example, wherein the user mistakes one sample for another, incorrectly locates operating instructions on a computer or within a guide or manual, misinterprets instructions, or incorrectly enters operating parameters.

Biological research reagents are often used in procedures that test a property or function of a sample. In many cases, a second test or procedure can be performed on a sample that is contingent on the results of a first test or procedure. Test results or procedure outcomes can be delayed while results are interpreted and the user again enters experimental parameters for a later analysis step. This process is also subject to misinterpretation and operator error, that can lead to the loss of time and sample.

There is a need for improved systems to handle, track, and label physical objects used in biological research, such as in a scientific research and development lab.

### BRIEF SUMMARY OF THE INVENTION

The invention provides biological research reagents comprising radio frequency identifier (RFID) tags that can streamline the use of the reagents in performing biological research procedures by allowing reagent, sample, and/or assay result-based information to be processed, recorded, and physically linked to the biological research reagent. The RFID tagging system can be used to manage samples and experimental workflow, or to direct steps of an analysis or processing procedure with reduced lag time during or between procedures. The invention can be used to reduce manual data entry and sample sorting for downstream analysis steps. The invention further provides systems for analyzing biological samples using biological research products that are associated with RFID tags, including for example, gels, chips, plates, and binding supports such as membranes or filters.

A first aspect of the invention is a biological research reagent associated with an RFID tag. The RFID tag associated with the biological research reagent can be a passive tag or an active tag, and preferably includes information such as, but not limited to, one or more of: 1) the identity of one or more components of the biological research reagent (biological research reagent identity information); 2) the type, quantity, concentration, or identity of a sample that is provided or is to be provided in or on the biological research product (sample identity information); and/or 3) one or more procedures that has been performed or is to be performed on a sample that is in or on the biological research product, is to be placed in or on the biological research reagent, or is to be synthesized in or on the biological research reagent (sample procedure information).

In some preferred embodiments, the invention includes a biological research reagent that has an associated RFID tag that includes both research reagent or sample identity information and sample procedure information. In some embodiments, the invention includes a biological research reagent that has an associated RFID tag that includes biological research product identity information, sample identity information, and sample procedure information. In some exemplary embodiments, a biological research reagent having an associated RFID tag is a biological research product that can contain, hold, or support a biological sample such as, for example, a gel or gel cassette, a gel strip, a filter or membrane, an array, a chip, or a plate, such as but not limited to a multiwell plate.

In another aspect, the present invention provides a method of tracking a biological research reagent that has an RFID tag associated with it. The biological research reagent can be a biomolecule (for example, one or more nucleic acids, one or more proteins, one or more antibodies, etc.) or reagents used for biological research, such as, for example, enzymes, cofactors, labeling molecules, nucleic acid vectors, etc.. Biological research reagent also includes cells, viruses, or cell extracts. The biological research reagent can also be a biological research product such as a vessel, substrate, separation medium or structure, or a structure, device, or apparatus for performing separations, detections, reactions, binding, assays, or biochemical syntheses.

In a further aspect, the present invention a system for performing at least one assay, separation, reaction, biochemical synthesis, or sample processing step, using a biological research reagent, in which the system includes at least one powered biological research device for performing an assay, detection, separation, reaction, biochemical synthesis, or sample processing step, an RFID tag reader, and a processing unit that can store the information read by the reader, and preferably link the reader information to information on the parameters or results of a procedure performed using the powered research device. The system uses at least one research reagent that includes an associated RFID tag to perform an assay, a detection, a separation, a reaction, a biochemical synthesis, or a sample processing step. The reader can receive reagent identity information, sample identity information, or sample procedure information stored on the RFID tag of the one or more research reagents.

In exemplary embodiments, a research reagent with an RFID tag used in these methods is a research product such as a gel, gel strip, or gel cassette, a filter or membrane, an array, a chip, or a plate; in which one or more assays, separations, syntheses, processing steps, or reactions can be performed in or on the research product. In other exemplary embodiments, a biological research reagent is provided in or on a container, support or structure that has an attached or embedded RFID tag. The powered research device can be, for example, an electrophoresis power supply or an optical scanner that can scan gels, filters, or arrays. In preferred embodiments, the processing unit of the system can integrate information from the RFID tag associated with a research product with results of an assay, detection, reaction, biochemical synthesis, or processing step performed by the system on a sample provided in or on the biological research product. In some embodiments, the processing unit can use information read from the tag and, in some preferred embodiments, obtained from results of procedures performed using the powered device, to access additional information entered by the user or provided in a linked database.

Yet another aspect of the invention is a biological research product that includes a writable RFID tag that includes information on the identity of one or more components of the biological research reagent (reagent identity information). The writable RFID tag on the biological research reagent has memory space that can accommodate additional information added during the course of the use of the research reagent by the user. For example, the user can write to the tag information on the type, quantity, concentration, or identity of a sample that is to be used in a procedure with the biological research reagent (sample identity information); or can write to the tag one or more procedures performed or to be performed on a sample with the biological research reagent.

Another aspect of the invention is a method of using a biological research reagent that includes an RFID tag in an experimental protocol, assay, or procedure, or in sample processing. The RFID tag includes one or both of information on the type, identity of the reagent; the type or identity of a sample to be used in a procedure with the biological reagent; or one or more procedures to be performed on a sample using the biological research reagent. In some preferred embodiments, the method includes reading the information on the RFID tag, in which the information provided on the RFID tag controls or directs at least one assay step, detection step, separation step, reaction, synthesis, or processing step that is performed on a sample using the biological research reagent. In some preferred embodiments, the method includes writing to an RFID tag associated with a biological research reagent information on or results of a procedure performed using the reagent.

In some exemplary embodiments, a research reagent or product with an associated RFID tag is a research product such as a gel or gel cassette, a filter or membrane, an array, a chip, or a plate, such as but not limited to a multiwell plate; in which one or more assays, detections, separations, syntheses, processing steps, or reactions can be performed in or on the research product, and information about the samples/and or procedures used with the reagent or product can be written to the RFID tag associated with the research product.

A further aspect of the invention is a system for performing at least one assay, reaction, biochemical synthesis, or processing step on a sample that is in or on a biological product that has an associated writable RFID tag. The system includes at least one RFID tag reader and a processor for converting the information read by the reader into stored information. The system further includes RFID tag writer, and, preferably, at least one detection or monitoring device. The reader can receive information stored on the RFID tag of the one or more research reagents. The information stored on an RFID tag read by the system reader is information concerning the research reagent, information concerning one or more samples associated with the research reagent; or information concerning one or more assays, detections, separations, reactions, syntheses or processing steps that has been performed on a sample associated with the research reagent or is to be performed on a sample associated with the research reagent. The RFID tag writer can write further information to the research product ID tag, such as information on the results of an assay, reaction, biochemical synthesis, or processing step as detected or monitored by the system.

A further aspect of the invention is a method of using a biological research product that includes a writable RFID tag, in which the method includes: performing one or more procedures on at least one sample that is in, on, attached to, or supported by the biological research product that includes a writable RFID tag; detecting, monitoring, or observing the results of the one or more procedures; and writing information based on the outcome of the one or more procedures on the writable RFID tag.

In some preferred embodiments, the method further includes: reading the outcome information encoded on the RFID tag associated with the research product using an RFID tag reader; and using the outcome information to direct at least one additional procedure on the sample. In preferred embodiments, these steps are automated, so that the results of a procedure are detected or monitored by a machine or device that communicates the results to a processing unit, and the processing unit communicates with an RFID tag writer to encode the result-based information on an RFID tag associated with the biological research product. In a further step, the result-based information is read by a reader that interfaces with a processing unit that directs a further analysis or processing step that is performed on the sample.

Yet another aspect of the invention is a method of obtaining information from a database based on an experimental result that is encoded on an RFID tag. The method includes: writing information based on the result or an experiment or test on a sample to an RFID tag associated with a biological research product, in which the biological research product holds or supports the sample; reading the information from the RFID tag, where the information is communicated to a processor that includes or is linked to a database; and obtaining information from the database that relates to the tested sample. The database can be, for example, a chemical structure database; sequence database, such as a database of nucleic acid or protein sequences, a database of biochemicals of any type, such as but not limited to: carbohydrates, steroids, lipids, small molecules, subclasses of molecules (e.g., mammalian kinases, mRNAs expressed in stem cells, etc.); or a scientific literature database or research agency database.

Another aspect of the invention is a set of two or more different research reagents with RFID tags, in which at least two of the set of the RFID tagged research reagents are read by an RFID reader that communicates information to a common central processing unit. In preferred embodiments, the different reagents of the set are used in a common workflow in testing or processing a sample.

### BRIEF DESCRIPTION OF THE DRAWINGS

FIG. 1 depicts a gel that includes an RFID tag, in which the RFID tag includes information on the composition of the gel that is read by a reader that interacts with software to direct electrophoresis conditions. Features of the drawing are not to scale.

FIG. 2 depicts a gel cassette that includes an RFID tag, in which the RFID tag includes information on the samples loaded on the gel that is read by a reader that interacts with software that also interfaces with a gel scanner to produce and store images produced by the gel scanner that are that include or are linked to sample information.

FIG. 3 depicts a) a functional chip that includes a readable/writable RFID tag, and B) a workflow for a system that uses the functional chip.

FIG. 4 is a diagram of a protein expression profiling workflow within a functional proteomics discipline. The first row of text boxes provides illustrative applications, the second row provides exemplary technologies and methods, and the third row provides exemplary products and tools. A) “PAGE/MS” can also include protein microarrays and mass spectrometry; “Cells” can also include tissue culture products and plasma; “Chromatography” includes Affinity chromatography, Ion exchange chromatography, Size-exclusion chromatography, Hydrophobic chromatography, and also protein precipitation; the “2D-PAGE” box also includes ID PAGE and HPLC, and the “Fusion proteins” box also includes stains and Western blots. The “Cells Products” box includes: Sf21 Cells, Drosophila S2 Cells, Sf9 Cells, Mimic Sf9 Insect Cells, 293 Cells, DH1OB Cells, DH5 Cells, and High Five Cells, SFM-adapted. “Protein Extraction products” include RELAY™ 96 Protein Screen (Invitrogen, Carlsbad, Calif.), System ZOOM® 2D protein solubilizers (Invitrogen, Carlsbad, Calif.) and related products: INVITROSOL™ MALDI Protein Solubilizer (Invitrogen, Carlsbad, Calif.), KITEZQ™ Protein Quantitation Kit (Invitrogen, Carlsbad, Calif.). “Affinity Purification Products” includes PROBOND™ Purification System (Invitrogen, Carlsbad, Calif.), Ni-NTA Purification System, Protein A Agarose, Recombinant Protein G (rProtein G) Agarose, Streptavidin Agarose, Glutathione-Agarose, and related products: DES® BIOEASE™ Expression Kit with pCoBlast, INVITROSOL™ (Invitrogen, Carlsbad, Calif.), MALDI Protein Solubilizer kit (Invitrogen, Carlsbad, Calif.). “2D PAGE products” includes the ZOOM® IPGRunner™ System (Invitrogen, Carlsbad, Calif.), ZOOM® Gels for 2D Electrophoresis (Invitrogen, Carlsbad, Calif.), ZOOM® Carrier Ampholytes (Invitrogen, Carlsbad, Calif.), ZOOM® Strips (Invitrogen, Carlsbad, Calif.), and related products: SIMPLYBLUE™ SafeStain (Invitrogen, Carlsbad, Calif.), SILVERQUEST™ Silver Staining Kit (Invitrogen, Carlsbad, Calif.), MULTIPLEXED PROTEOMICS® Phosphoprotein Gel Stain Kit #1 (Invitrogen, Carlsbad, Calif.), SYPRO® Ruby protein gel stain (Invitrogen, Carlsbad, Calif.), PRO-Q® Diamond phosphoprotein gel stain (Invitrogen, Carlsbad, Calif.), and SYPRO® Tangerine protein gel stain (Invitrogen, Carlsbad, Calif.). “Fusion Protein Products” include: LUMIO™ Green Detection system, KITINVISION™ His-tag In-gel Stain, CHAMPION™ pET Expression System with LUMIO™ Technology, and the Expressway Plus Expression System with LUMIO™ Technology, all from Invitrogen (Carlsbad, Calif.). B) “Nonlinear Dynamics” (leftmost box in the middle row) includes Decodon and GeneBio. The “Protein Expression Software” box in the middle row also includes other software or MapWeb. The Microarrays box in the middle row also includes mass spectrometry. “Functional Proteomics” can also include protein-protein interaction, and “Functional Genomics” can include gene expression profiling and cloning. “Drug Discovery” includes biomarker analysis. The box “Recommended products from Linear Dynamics” includes the following software programs: Phoretix 2D, Phoretix 2D Expression, Phoretix 2D Evolution, Progenesis Workstation, Progenesis Discovery. “Protein Expression Software” includes Vector Xpression, GeneSpring, and Acuity. “In-gel Digestion Products” include INVITROSOL™ MALDI Protein Solubilizer kit (Invitrogen, Carlsbad, Calif.). MALDI-MS products include INVITROSOL™ MALDI Protein Solubilizer kit (Invitrogen, Carlsbad, Calif.), INVITROMASS™ High MW Mass Calibrant kit (Invitrogen, Carlsbad, Calif.), and INVITROMASS™ Low MW Mass Calibrant kit (Invitrogen, Carlsbad, Calif.).

### DETAILED DESCRIPTION OF THE INVENTION

### Definitions

Unless defined otherwise, all technical and scientific terms used herein have the same meaning as commonly understood by one of ordinary skill in the art to which this invention belongs.

Where a term is provided in the singular, the inventors also contemplate the plural of that term. As employed throughout the disclosure, the following terms, unless otherwise indicated, shall be understood to have the following meanings:

The term “biological reagents” as used herein generally refers to isolated biomolecules and biological research products utilized in biological research procedures.

“Biomolecules” include but are not limited to various classes of biomolecules, including proteins, peptides, antibodies, nucleic acids, nucleotides, lipids, steroids, polysaccharides, carbohydrates, and variants of the foregoing, for example. For example, nucleic acids can include, but are not limited to, open reading frames, structural genes, or transcription units. Two target biomolecules are “different” when they are structurally different. For example, two different nucleic acids have different nucleotide sequences. Two different proteins have different amino acid sequences. Biomolecules may be categorized into families or subclasses based on, for example, a function of the related protein or nucleic acid, such as the functions of the proteins or, for example, based on the activity of the related protein or nucleic acid, such as those having enzyme classifications (for illustrative purposes only, a protein kinase family may have various subclasses of protein kinases, such as, for example, tyrosine kinases and serine/threonine kinases, each subclass can itself be further subdivided into narrower subclasses).

The term “biological research product” is used to mean a product that is used in biological research. Virtually every product available from a biological research product vendor such as Invitrogen (Carlsbad, Calif.; invitrogen.com) is included in the term biological research product”. Biological research products include various types of biological research products, protocols, instruments, and services, including, but not limited to, products such as, for example, cell culture products, labeling reagents, detection products, separation media and systems, and microarrays, for example; services, such as, for example, nucleic acid synthesis, protein synthesis, vector construction, and performance of one or more assays; protocols, reagents, and kits for biochemical procedures such as, for example, constructing a vector, transforming or transfecting cells; performing an assay, synthesizing nucleic acids or proteins, or making a monoclonal antibody; or apparatuses or instruments such as electrophoresis apparatuses, mass spectrometers, microscopes, or microfluidic devices. Further examples of biological research products include but are not limited to gels, enzymes, buffers, substrates, cofactors, indicator molecules, bioassays, vectors, molecular weight markers, synthetic nucleic acids (e.g., DNA and RNA primers and pairs of primers), cloning reagents, PCR reagents, cell culture products, and kits reagents needed for bioassays and syntheses. In some aspects, preferred biological research products include vessels, matrices, supports, or other structures that can contain, hold, or support a sample. For example, gels, gel or matrix strips, cassettes (such as cassettes that hold gels for electrophoresis), columns, tubes, vials, plates (such as but not limited to multiwell plates), chips, arrays, membranes, or filters are some preferred biological research products.

A biological research product or isolated biomolecule, can include, for example, any of the biological research products, services, instruments, protocols, or isolated biomolecules in the collection of biological research products, services, protocols, instruments, and isolated biomolecules available from a commercial biological research reagent, service, and/or instrument provider. A biological research product or isolated biomolecule, can include, for example, any of the biological research products, services, protocols, or isolated biomolecules in the collection of biological research products, services, protocols, and isolated biomolecules disclosed at and linked to the Internet site available on the worldwide web at the URL invitrogen.com, which Internet site is incorporated by reference in its entirety on the date this application is filed, and available in the 2005 catalog of Invitrogen Corporation (Carlsbad, Calif.), which is incorporated by reference in its entirety on the date this application is filed, the 2005 catalog of Dynal Biotech (Oslo, Norway), which is incorporated by reference in its entirety on the date this application is filed, the 2005 catalog of Zymed, Inc. (South San Francisco, Calif.), and the 2005 catalog of BioSource International, Inc. (Camarillo, Calif.), which is incorporated by reference in its entirety on the date that this application is filed.

“Matched biological reagents” include the following: (i) two or more isolated biomolecules that relate to the same gene; (ii) a combination of one or more isolated biomolecules that relate to the same gene and one or more biological research products that are used to study the gene, (iii) biological research products that are used to study a class of biomolecules and/or a sub-class of biomolecules and optionally one or more isolated biomolecules of the class of biomolecules and/or sub-class of biomolecules and that relate to the same gene, (iv) biological research products that are used in the same or subsequent steps of a workflow and optionally one or more isolated biomolecules studied using the workflow and that relate to the same gene, and (v) biological research products that are used to study a disease and optionally isolated biomolecules that are involved in the disease, such as isolated biomolecules involved in a pathway of the disease. A set of matched biological reagents includes more than one type of matched biological reagent. Fifty sets of matched biological reagents, for example, can include 50 isolated proteins, 50 nucleic acids each encoding a different one of the 50 isolated proteins, and 50 antibodies each recognizing a different one of the isolated proteins. In this example, 3 classes of biomolecules make up one set of matched reagents. The sets, in this example, can be further expanded to include, for example, biological research products, such as 2 types of biological research products. The biological research products can be, for example, research products that are used to analyze proteins (e.g., protein gels) and/or research products that are used to analyze nucleic acids (nucleic acid gels) and/or research products that include antibodies (enzyme-linked immunoassay kits). Accordingly, different matched reagent sets can include the same research products. A collection of matched biological reagents includes one or more sets of matched biological reagents.

A biological research kit is a collection of biological research products that are used to perform a biological research reaction, procedure, or synthesis, such as, for example, a detection, assay, separation, purification, etc., which are typically shipped together, usually within a common packaging, to an end user.

The terms “polynucleotide” or “nucleic acid molecule” are used broadly herein to mean a sequence of two or more deoxyribonucleotides or ribonucleotides that are linked together by a phosphodiester bond. As such, the term “polynucleotide” or “nucleic acid molecule” includes RNA and DNA, which can be a synthetic RNA or DNA sequence, and can be single stranded or double stranded, as well as a DNA/RNA hybrid. Furthermore, the term “polynucleotide” or “nucleic acid molecule” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR). In various embodiments, a polynucleotide or nucleic acid molecule useful according to the present invention can contain nucleoside or nucleotide analogs, or a backbone bond other than a phosphodiester bond.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., NucL Acids Res. 22:5220-5234, 1994; Jellinek et al., Biochemistry 34:11363-11372, 1995; Pagratis et al., Nature Biotechnol. 15:68-73, 1997, each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide or peptide-like bond or any other bond known to those in the art as useful for linking nucleotides to produce synthetic polynucleotides (see, for example, Tam et al., NucL Acids Res. 22:977-986, 1994; Ecker and Crooke, BioTechnology 13:351360, 1995, each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

The term “peptide” is used broadly herein to mean two or more amino acids linked by a peptide bond. Generally, a peptide useful in the invention contains at least about two, three, four, five, or six amino acids, and can contain about ten, fifteen, twenty or more amino acids. As such, it should be recognized that the term “peptide” is not used herein to suggest a particular size or number of amino acids comprising the molecule, and that a peptide of the invention can contain up to several amino acid residues or more. As used herein, the term “polypeptide” refers to a sequence of contiguous amino acids of any length where the amino acids are attached by peptide bonds. The terms “oligopeptide,” or “protein” may be used interchangeably herein with the term “polypeptide.” The terms “peptide”, “protein”, or “polypeptide” includes peptides and proteins that comprise or linked to carbohydrate moieties, lipid, phosphate groups, labels, etc.

A peptide of the invention can be prepared, for example, by a method of chemical synthesis, or can be expressed from a polynucleotide using recombinant DNA methodology. Where chemically synthesized, peptides containing one or more D-amino acids, or one or more amino acid analogs, for example, an amino acid that has been derivatized or otherwise modified at its reactive side chain, or in which one or more bonds linking the amino acids or amino acid analogs is modified, can be prepared. In addition, a reactive group at the amino terminus or the carboxy terminus or both can be modified. Such peptides can be modified, for example, to have improved stability to a protease, an oxidizing agent or other reactive material the peptide may encounter in a biological environment, and, therefore, can be particularly useful in performing a method of the invention. Of course, the peptides can be modified to have decreased stability in a biological environment such that the period of time the peptide is active in the environment is reduced.

“Specific binding member” is one of two different molecules having an area on the surface or in a cavity which specifically binds to and is thereby defined as complementary with a particular spatial and polar organization of the other molecule. A specific binding member can be a member of an immunological pair such as antigen-antibody, biotin-avidin, hormone-hormone receptor, nucleic acid duplexes, IgG-protein A, DNA-DNA, DNA-RNA, and the like.

As used herein, the term “antibody” is used in its broadest sense to include polyclonal and monoclonal antibodies, as well as antigen binding fragments of such antibodies. The term “binds specifically” or “specific binding activity,” when used in reference to an antibody means that an interaction of the antibody and a particular epitope has a dissociation constant of at least about 1×10⁻⁶ M, generally at least about 1×10⁴ M, usually at least about 1×10⁻⁸ M, and particularly at least about 1×10⁻⁹ M or 1×10⁻¹⁰ M or less. As such, Fab, F(ab′)2, Fd and Fv fragments of an antibody that retain specific binding activity for an epitope of a polypeptide, are included within the definition of an antibody.

An antibody of the invention additionally includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281 (1989), which is incorporated herein by reference). These and other methods of making, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995); each of which is incorporated herein by reference).

Methods for raising polyclonal antibodies, for example, in a rabbit, goat, mouse or other mammal, are well known in the art (see, for example, Green et al., “Production of Polyclonal Antisera,” in Immunochemical Protocols (Manson, ed., Humana Press 1992), pages 1-5; Coligan et al., “Production of Polyclonal Antisera in Rabbits, Rats, Mice and Hamsters,” in Curr. Protocols Immunol. (1992), section 2.4.1; each or which is incorporated herein by reference). In addition, monoclonal antibodies can be obtained using methods that are well known and routine in the art (Harlow and Lane, supra, 1988). Methods of preparing monoclonal antibodies well known (see, for example, Kohler and Milstein, Nature 256:495, 1975, which is incorporated herein by reference; see, also, Coligan et al., supra, 1992, see sections 2.5.1-2.6.7; Harlow and Lane, supra, 1988).

Monoclonal antibodies can be isolated and purified from hybridoma cultures by a variety of well established techniques, including, for example, affinity chromatography with Protein-A SEPHAROSE gel, size exclusion chromatography, and ion exchange chromatography (Coligan et al., supra, 1992, see sections 2.7.1-2.7.12 and sections 2.9.1-2.9.3; see, also, Barnes et al., “Purification of Immunoglobulin G (IgG),” in Meth. Molec. Biol. 10:79-104 (Humana Press 1992), which is incorporated herein by reference).

Antibodies of the invention also can be derived from human antibody fragments isolated from a combinatorial immunoglobulin library (see, for example, Barbas et al., METHODS: A Companion to Methods in Immunology 2:119, 1991; Winter et al., Aim. Rev. Immunol. 12:433, 1994; each of which is incorporated herein by reference). Cloning and expression vectors that are useful for producing a human immunoglobulin phage library can be obtained, for example, from STRATAGENE Cloning Systems (La Jolla, Calif.).

An antibody of the invention also can be derived from a human monoclonal antibody. Such antibodies are obtained from transgenic mice that have been “engineered” to produce specific human antibodies in response to antigenic challenge. Methods for obtaining human antibodies from transgenic mice are described, for example, by Green et al., Nature Genet. 7:13, 1994; Lonberg et al., Nature 368:856, 1994; and Taylor et al., Int. immunol. 6:579, 1994; each of which is incorporated herein by reference.

“Organism” can be any prokaryote or eukaryote, and includes viruses, bacteria, protozoans, and metazoans. Metazoans include vertebrates and invertebrates. “Organism” can also refer to more than one species that are found in association with one another, such as mycoplasm-infected cells, a plasmodium-infected animal, etc.

A “probe” or “probe nucleic acid molecule” is a nucleic acid molecule that is at least partially single-stranded, and that is at least partially complementary, or at least partially substantially complementary, to a sequence of interest. A probe can be RNA, DNA, or a combination of both RNA and DNA. It is also within the scope of the present invention to have probe nucleic acid molecules comprising nucleic acids in which the backbone sugar is other that ribose or deoxyribose. Probe nucleic acids can also be peptide nucleic acids. A probe can comprise nucleolytic-activity resistant linkages or detectable labels, and can be operably linked to other moieties, for example a peptide.

A single-stranded nucleic acid molecule is “complementary” to another single-stranded nucleic acid molecule when it can base-pair (hybridize) with all or a portion of the other nucleic acid molecule to form a double helix (double-stranded nucleic acid molecule), based on the ability of guanine (G) to base pair with cytosine (C) and adenine (A) to base pair with thymine (T) or uridine (U). For example, the nucleotide sequence 5′-TATAC-3′ is complementary to the nucleotide sequence 5′-GTATA-3′.

“Substantially complementary” refers to nucleic acids that will selectively hybridize to one another under stringent conditions. “Selectively hybridize” refers to detectable specific binding. Polynucleotides, oligonucleotides and fragments thereof selectively hybridize to target nucleic acid strands, under hybridization and wash conditions that minimize appreciable amounts of detectable binding to nonspecific nucleic acids. High stringency conditions can be used to achieve selective hybridization conditions as known in the art Generally, the nucleic acid sequence complementarity between the polynucleotides, oligonucleotides, and fragments thereof and a nucleic acid sequence of interest will be at least 30%, and more typically and preferably of at least 40%, 50%, 60%, 70%, 80%, 90%, and can be 100%. Conditions for hybridization such salt concentration, temperature, detergents, and denaturing agents such as formamide can be varied to increase the stringency of hybridization, that is, the requirement for exact matches of C to base pair with G, and A to base pair with T or U, along the strand of nucleic acid.

A “detectable label” is a compound or molecule that can be detected, or that can generate a readout, such as fluorescence, radioactivity, color, chemiluminescence or other readouts known in the art or later developed. The readouts can be based on fluorescence, such as by fluorescent labels, such as but not limited to, Alexa compounds, Cy-3, Cy-5, phycoerthynin, phycocyanin, allophycocyanin, FITC, rhodamine, or lanthanides or fluorescent variants or derivatives of any of these; by flourescent proteins such as green fluorescent protein (GFP) and its variants, can be based on enzymatic activity, such as, but not limited to, the activity of beta-galactosidase, beta-lactamase, GUS, horseradish peroxidase, alkaline phosphatase, or luciferase; or can be based on radioisotopes (such as ³³P, ³H, ¹⁴C, 35S, 125_{1,} 32., or ¹³¹1). I). A label optionally can be a base with modified mass, such as, for example, pyrimidines modified at the C5 position or purines modified at the N7 position. Mass modifying groups can be, for examples, halogen, ether or polyether, alkyl, ester or polyester, or of the general type XR, wherein X is a linking group and R is a mass-modifying group. One of skill in the art will recognize that there are numerous possibilities for mass-modifications useful in modifying nucleic acid molecules and oligonucleotides, including those described in Oligonucleotides and Analogues: A Practical Approach, Eckstein, ed. (1991) and in PCT/US94/00193, herein incorporated by reference for all disclosure of nucleotides, oligonucleotides, and their modifications.

“Label” or “labeled” refers to incorporation of a detectable marker, for example by incorporation of a fluorescent or radiolabled compound or attachment of moieties such as biotin that can be detected by the binding of a second moiety, such as labeled avidin. Various methods of labeling nucleic acids are known in the art

A “mutation” is a change in the genome with respect to the standard wild-type sequence. Mutations can be deletions, insertions, or rearrangements of nucleic acid sequences at a position in the genome, or they can be single base changes at a position in the genome, referred to as “point mutations”. Mutations can be inherited, or they can occur in one or more cells during the lifespan of an individual.

“Operably linked” refers to a juxtaposition wherein the components so described are in a relationship permitting them to function in their intended manner For example, a control sequence operably linked to a coding sequence is positioned in such a way that expression of the coding sequence is achieved under conditions compatible with control sequences.

A “solid support” is a solid material having a surface for attachment or support of molecules, compounds, cells, or other entities. The surface of a solid support can be flat or not flat. A solid support can be porous or non-porous. A solid support can be a chip or array that comprises a surface, and that may comprise glass, silicon, nylon, polymers, plastics, ceramics, or metals, including laminated metal. A solid support can also be a membrane, such as a nylon, nitrocellulose, PVDF or other polymeric membrane, or a plate or dish and can be comprised of glass, ceramics, metals, or plastics, such as, for example, a 96-well plate made of, for example, polystyrene, polypropylene, polycarbonate, or polyallomer. A solid support can be a gel or matrix that comprises gelatin, starch, agarose, acrylamide, sepharose, cellulose or cellulose derivatives, etc. A solid support can also be a bead or particle of any shape. Such particles or beads can be comprised of any suitable material, such as glass or ceramics, and/or one or more polymers, such as, for example, nylon, polytetrafluoroethylene, TEFLON™, polystyrene, polyacrylamide, sepaharose, agarose, cellulose, cellulose derivatives, or dextran.

A “functional chip” is a surface on which at least one assay, reaction, or process can be performed. A chip can be a solid or semisolid substrate, porous or non-porous on which certain processes, such as physical, chemical, biological, biophysical or biochemical processes, etc., can be carried out. A chip that performs more than one function can have combinations of one or more different functional elements such specific binding members, substrates, reagents, or different types of micro-scale structures that provide sources of different physical forces used in the processes carried out on the chip. Micro-scale structures such as but not limited to channels and wells, electrode elements, electromagnetic elements, and piezoelectric transducers can be incorporated into, fabricated on, or otherwise attached to the chip substrate for facilitating physical, biophysical, biological, biochemical, or chemical reactions or processes on the chip. The chip may be thin in one dimension and may have various shapes in other dimensions, for example, a rectangle, a circle, an ellipse, or other irregular shapes. The size of the major surface of chips of the present invention is not limiting and can vary considerably, e.g., from about 1 mm² to about 100 cm². Preferably, the size of the chips is from about 4 mm² to about 25 cm² with a characteristic dimension from about 1 mm to about 5 cm. Chip surfaces may be flat, or not flat. Chips with non-flat surfaces may include channels or wells fabricated on the surfaces. Examples of functional chips include those described in U.S. Pat. Nos. 6,881,314; 6,596,143; 6,858,439, 6,806,050; 6,071,394;6,280,590; 6,942,778; 6,887,362; 6,867,048; and 6,824,740, all of which are herein incorporated by reference in their entireties for their disclosure of functional chips, methods of making functional chips, and method of using functional chips. The chip, in certain embodiments is a microarray that includes at least 100 biomolecules/cm².

“Micro-scale structures” are structures integral to or attached on a chip or chamber for sample testing and processing that have characteristic dimensions of scale for use in microfluidic applications ranging from about 0.1 micron to about 20 mm. Example of micro-scale structures are wells, channels, scaffolds, electrodes, electromagnetic units, piezoelectric transducers, metal wires or films, Peltier elements, microfabricated pumps or valves, microfabricated capillaries or tips, or optical elements. A variety of micro-scale structures are disclosed in U.S. Pat. Nos. 6,858,439; 6,881,314; 6,596,143; and 6,071,394; herein incorporated by reference in their entireties for all disclosure of microscale structures on chips, their manufacture and use. Micro-scale structures that can, when energy, such as an electrical signal, is applied, generate physical forces useful in the present invention, can be referred to as “physical force-generating elements” “physical force elements”, “active force elements”, or “active elements”.

A “radio frequency identifier tag” or “RFID tag” is a chip containing an integrated circuit attached to antenna. Sometimes herein, a radio frequency identifier (RFID) tag is referred to as a radio frequency identifier or RFID, wherein the fact that the RFID is a tag is implied. The integrated circuitry stores data (typically encoding an identifier) that can be communicated by a radio frequency transmitted by the antenna. Typically the integrated circuit and antenna circuitry are printed on the chip. An RFID “tag” or “transponder” can be read by an RFID reader that also has an antenna that emits radio frequencies to query the transponder. A “passive RFID” does not have its own energy source, but responds to signals from a reader to transmit a signal. An “active RFID” includes a battery as a power source. The battery can increase the effective range or the functional capacity of the RFID tag. Some examples of RFID tags can be found in U.S. Pat. Nos. 6, 147,662; 6,917,291; 5,949,049; 6,652,812; 6,112,152; and U.S. Patent Application No. 2003/0183683 all of which are herein incorporated by reference in their entireties for their disclosure of RFID tags, chips, labels, or devices, RFID readers, and RFID systems, their design and use.

A “writable radio frequency identifier” or “writable RFID” is an RFID tag that has memory space that can be written to by an RFID writer.

An “assay” can be any type of assay, including without limitation, detection assays, such as but not limited to binding assays, functional assays, such as but not limited to enzymatic assays, ion transport assays, GPCR assays, gene expression assays, or cellular assays such as apoptosis assays or a cell migration assay. including, without limitation, cell separation, cell purification, biomolecule separation, biomolecule purification.

A “processing step” is any procedure in the processing of a sample, including a separation step (for example, based on size, isoelectric point, or binding affinity or specifity of sample components), an amplification step (for example, PCR), a concentration step, a mixing step, or a step that includes structural alteration of one or more sample components (for example, a lysis step, solubilization step, or denaturation step).

A “biochemical synthesis” is a procedure in which one or more organic molecules is synthesized. A synthesis can in some cases require multiple steps and/or multiple reagents. Examples of biochemical syntheses are those performed by polymerase reactions such as but not limited to PCR, reverse transcription, and transcription; protein translation; peptide synthesis; chemical conjugations (such as but not limited to, the addition of labels); etc.

A “sample” is any material or substance that is to be assayed for the presence or activity of one or more molecules or complexes of interest, used for the synthesis of one or more compounds, or used for the separation, isolation, or purification of one or more molecules or complexes. In preferred aspects, a sample is an environmental sample (for example, a soil sample, a water sample) or a biological sample. As used herein, a biological sample also includes any sample that includes one or more biomolecules in unpurified, partially purfied, or substantially purified form. A biological sample can be, for example, a suspension of cells of any type; a sample of a bodily fluid, including blood, urine, saliva, etc.; a cell extract; a cell fraction; partially or substantially purified biomolecules such as but not limited to nucleic acids or proteins; etc. The term biological sample also includes biochemical samples that include biomolecules that may have been isolated from cells or viruses or chemically synthesized, such as, for example, nucleic acids, nucleotides, peptides, amino acids, carbohydrates, lipids, steroids, and the like.

As used herein, “biomolecules” includes organic molecules of cellular and viral origin as well as synthetic organic molecules that are variants, derivatives, or combinations of naturally occurring biomolecules, or engineered biomolecules such as engineered nucleic acids and proteins. Also included are biomolecules derived from or based on naturally occurring biomolecules that include non-naturally occurring chemical moieties or groups, such as but not limited to, metals, tags or labels that can be used for binding or detection of molecules.

An expression vector (or the polynucleotide) generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, I Bioenerg. Biomemb. 25:37-42, 1993; Kirshenbaum et al., J. Clin. Invest. 92:381-387, 1993; each of which is incorporated herein by reference).

A polynucleotide, which can be contained in a vector, can be introduced into a cell by any of a variety of methods known in the art (Sambrook et al., Molecular Cloning: A laboratory manual (Cold Spring Harbor Laboratory Press 1989); Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. The selection of a particular method will depend, for example, on the cell into which the polynucleotide is to be introduced, as well as whether the cell is isolated in culture, or is in a tissue or organ in culture or in situ.

The present invention further provides storage vessels, containers, etc. having an RFID tag associated therewith and containing a cell or a plurality of cells or medium for growing cells. The term “cell” refers generally to a small compartment or bounded space including, for example, a small mass of protoplasm bounded externally by a semipermeable membrane, usually including one or more nuclei and various nonliving products, capable alone or interacting with other cells of performing all the fundamental functions of life, and forming the smallest structural unit of living matter capable of functioning independently. Thus, a cell is generally a biological cell, and includes, without limitation, any prokaryotic, eukaryotic, bacterial, fungal, animal, plant, algae cell or otherwise. As such, the cell can be a eukaryotic cell, including, for example, an insect cell (e.g., a Drosophila cell), a fungus cell (e.g., a Neurospora cell), a yeast cell, a C. elegans cell, an amphibian cell (e.g., sea urchin), an avian cell (e.g., a chick embryo fibroblast), or a human cell (e.g., a human T lymphocyte). Further, such cells contained in a storage vessels, containers, etc. having an associated RFID tag of the invention can be cells of a cell line, which have been adapted to culture; can be cells of a primary cell culture, which can be maintained in culture for at least a short period of time; or cells that have been isolated from a living organism, for example, cells isolated from a human subject.

For administration to a living subject, the agent generally is formulated in a pharmaceutical composition suitable for administration to the subject. Thus, the present invention further provides storage vessels, containers, etc. having an associated RFID tag and containing pharmaceutical compositions containing an agent in a pharmaceutically acceptable carrier. As such, the agents are useful as medicaments for treating a subject suffering from a pathological condition as defined herein.

Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second reagent such as a diagnostic reagent, nutritional substance, toxin, or therapeutic agent, for example, a cancer chemotherapeutic agent.

The agent can be incorporated within an encapsulating material such as into an oil-inwater emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a pharmaceutical composition useful for practicing a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest., 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference).

The route of administration of a pharmaceutical composition containing an agent will depend, in part, on the chemical structure of the molecule. Polypeptides and polynucleotides, for example, are not particularly useful when administered orally because they can be degraded in the digestive tract. However, methods for chemically modifying polypeptides, for example, to render them less susceptible to degradation by endogenous proteases or more absorbable through the alimentary tract are well known (see, for example, Blondelle et al., supra, 1995; Ecker and Crook, supra, 1995). In addition, a peptide agent can be prepared using D-amino acids, or can contain one or more domains based on peptidomimetics, which are organic molecules that mimic the structure of peptide domain; or based on a peptoid such as a vinylogous peptoid.

A pharmaceutical composition as disclosed herein can be formulated for administration to an individual by various routes including, for example, orally or parenterally, such as intravenously, intramuscularly, subcutaneously, intraorbitally, intracapsularly, intraperitoneally, intrarectally, intracistemally or by passive or facilitated absorption through the skin using, for example, a skin patch or transdermal iontophoresis, respectively. Furthermore, the pharmaceutical composition can be formulated for administration by injection, intubation, orally or topically, the latter of which can be passive, for example, by direct application of an ointment, or active, for example, using a nasal spray or inhalant, in which case one component of the composition is an appropriate propellant. A pharmaceutical composition also can be formulated for administration to the site of a pathologic condition, for example, intravenously or intraarterially into a blood vessel supplying a tumor.

The pharmaceutical composition can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

All references provided herein, including published literature, patents, patent applications, and materials available on the world wide web are herein incorporated by reference in their entireties.

Headings are for the convenience of the reader only, and do not limit the invention in any way.

RFID-labeled biological research reagents and products

The present invention utilizes Wi-fi or Radio frequency identification tags. Wi-fi tags are available and known in the art (e.g., available from Ekahau (T101 Wi-fi tag), for more information see Ekahau.com or rfidjournal.com available on the worldwide web). For additional information regarding RFID technology, see the RFID White Papers available on the Worldwide web at Zebra.com. The RFID White Papers available at Zebra.com are incorporated herein in their entirety. Radio frequency identification is sometimes called dedicated short range communication (DSRC).

Radio frequency identification is a technology that incorporates the use of electromagnetic or electrostatic coupling in the radio frequency (RF) portion of the electromagnetic spectrum to uniquely identify an object, animal, or person. Radio frequency identification is coming into increasing use in industry as an alternative to the bar code. A major advantage of radio frequency identification is that it does not require direct contact or line-of-sight scanning A radio frequency identification system consists of three components: an antenna and transceiver (often combined into one reader) and a transponder (the tag), which includes an antenna and an integrated circuit (IC) for storing encoded information. The antenna uses radio frequency waves to transmit a signal that activates the transponder. When activated, the tag uses its antenna to transmit data back to the reader.

RFID tags are available with different memory sizes and encoding options. Typically, RFID tags for use with the present invention are of a size that is appropriate for the object with which they are associated. For example, the RFID can have an area of 1 cm-1 m, or a diameter of 1 mm to 1 M. RFID tags used in the present invention can either be passive (no battery) or active (self-powered by a battery). The additional power of an active RFID tag can increase the range at which the signal can be read and can increase the functionality of an RFID tag. In illustrative embodiments, a passive RFID tag is used. In some other embodiments of the invention, active RFID tags are used. In certain aspects, active tags are used that transmit a signal periodically.

Data transmission speed and range of the RFID tags used in the present invention can be determined based on the particular research reagent, and can be set by varying the radio frequency, antenna size, power output, and interference. Tags associated with research reagents in the present invention can be read-only, read-write, or a combination, in which some data (such as a serial number) is permanently stored (non-erasable), while other memory is left available for later encoding or to be updated (erased and rewritten) during usage.

The invention provides biological research reagents that include associated radio frequency identifier (RFID) tags. The associated RFID tags typically include information on at least one of: 1) the identity of one or more components of the biological research reagent and/or the kit that includes the biological research reagent (biological research reagent identity information); 2) the type, quantity, concentration, or identity of a sample that is provided or is to be provided in or on the biological research product (sample identity information); and 3) one or more procedures that has been performed or is to be performed on a sample that is in or on the biological research product, is to be placed in or on the biological research reagent, or is to be synthesized in or on the biological research reagent (sample procedure information).

The biological research reagent can be any research reagent used in biological science. For example, the biological research reagent can be a solution that includes a polypeptide, a nucleic acid, a polysaccharide, a dye, a stain, a co-factor, detection reagent, or a buffer used in biological research. Biological research reagents include biomolecules such as, for example, nucleic acids, proteins, antibodies, enzymes, as well as biological research products such as vectors, cells, cell extracts, virus preparations, cofactors, cell culture reagents, matrices, gels, columns, fractionators, plates, arrays, cassettes, etc.

An RFID tag is associated with the research reagent either by being irreversibly or reversibly attached to or embedded in a container that holds, supports, or contains the research reagent, or by being irreversibly or reversibly attached to or embedded in the research reagent itself. For example, reagents provided as dry chemicals or solutions can have RFID smart labels attached to tubes or vials that contain the chemicals or solutions, or embedded in the containers. In another example, a research reagent is a product that itself has an attached or embedded RFID tag. The research product can be, as nonlimiting examples, a filter, membrane, plate, slide, array, chip, column, cassette, gel, or gel strip.

In preferred embodiments, the RFID-labeled research reagent has at least one associated RFID tag that includes reagent or product-identifying information, such as, for example, a code that can be linked to the product name, kit name that includes the research reagent, and/or part number through a processer or computer that interfaces with an RFID tag reader. Preferably the lot number of the reagent or product is also encoded on the tag, and preferably the tag also includes a unique identifier corresponding to the individual item the tag is associated with. In certain aspects, biological sequence information, such as nucleic acid sequence information is read or written on an RFID tag.

In other embodiments, the invention includes a biological research reagent that has an associated RFID tag that includes readable information that includes additional biological research reagent identity information, such as, for example, information on the identity, concentration, or amount of a compound that makes up at least a part of the research reagent. A gel, for example, may have associated with it an RFID that encodes information on the gel buffer composition and acrylamide concentration.

For the present invention, an RFID is associated with a biological research reagent by associating an RFID tag (a transponder) with an object that contains a biological reagent. RFID tags typically consist of an integrated circuit (IC) attached to an antenna, which can be printed or etched conductors on a thin plastic sheet. Data is stored on the IC and transmitted through the antenna. A research reagent can be associated with an RFID tag by contacting the RFID tag with a vessel that includes the research reagent or embedding the RFID tag within a vessel that includes the research reagent. Research reagents with an associated RFID tag are also referred to as RFID-tagged or RFID-labeled research reagents herein. In RFID-tagged research reagents provided herein, the IC and antenna are typically associated with a biological research reagent.

The invention is based, in part, on the discovery that by associating an RFID tag with a research reagent, numerous advantages are realized that can improve the accuracy, security, and speed of research performed using the research reagent. Radio frequency identification technology provides numerous advantages for labeling objects over traditional technologies used for tracking physical objects such as barcodes, many of which are particularly advantageous in the context of scientific research and development, especially biotechnology laboratory research. As a radio technology, RFID requires no line-of-sight between the reader and the tag to exchange data. RFID tags therefore can be read through packaging, including cardboard containers and plastic wrap used to seal pallets. RFID is subject to interference however, particularly from metal, so potential sources of interference must be recognized and accounted for during system planning

Because no line-of-sight is required, tagged objects can be read regardless of their orientation through the use of optimized RFID systems. Product handlers can be more productive because they don't have to locate and align labels when handling research products. RFID readers can automatically recognize and differentiate all the RF tags in their reading field. This simultaneous processing capability provides additional flexibility for material handling, packaging and sorting operations because there is no need to maintain spacing between objects to ensure they will be read.

The data capacity of RFID tags enables them to carry all the same information as bar codes as well as additional information: For example, in addition identifying the type of reagent, a research product tag can include information on the expiration date of the reagent, and samples or other reagents it can be used with.

Accordingly, the present invention provides a radio frequency identifier (RFID)-labeled (or Wi-Fi-labeled) research reagent, having an RFID (or Wi-Fi) tag associated therewith. The research reagent can be a research reagent or product used in any scientific discipline. The research reagent is typically used in a government, academic, or industrial laboratory performing “wet lab” experiments, or analyzing data from a “wet lab.” For example, the research reagent can be a chemical or biological research reagent. In illustrative examples, the research reagent is a biological research reagent, which can be for example, any biological research product available from Invitrogen, as provided on the worldwide web at Invitrogen.com, incorporated &rein by reference.

In certain illustrative aspects, the RFID-tagged biological research reagent does not contain, hold, or support a biological sample collected from a subject, and/or is not contained within a sample collection container, such as a patient sample collection tube or bag. In other aspects, the biological research reagent is not a vial of a pharmaceutical drug in an approved form or a form used in FDA clinical trials. However, the present invention, in certain aspects includes a research product associated with an RFID, wherein the research reagent molecule includes a small organic molecule, such as a small organic molecule that is being tested in pre-clinical research. The biological research reagent can be associated with a bottle, a tube, a vial, a slide, a chip, an array, a bead, a particle, a column, a filter or membrane, a gel, a,gel cassette, or a plate. The tube can be, for example, a standard test tube, a tube used in fraction collection, or a tube for centrifugation, such as a microfuge tube or an ultracentrifuge tube. Typically, for the present invention the research reagent is contained or embedded within a vessel or support or localized on a surface of an object.

A research reagent in the form of a chemical, enzyme, solution, extract, etc. that has an associated RFID tag is preferably provided in or on a vessel or support that has an attached or embedded RFID tag. For example, a solution comprising a cofactor for a reaction can be in a vial or tube that has an RFID tag attached to the outside surface or embedded in the lid of the container. The attachment of labels to plates, tubes, vials, cartons, or packets that contain, hold, or support one or more biological research reagents can establish association of the RFID with the reagent.

The RFID-labeled (i.e. RFID-tagged) research reagent provided herein can be associated with a biological product in virtually any manner that can be used to attach a physical object with the properties of an RF identifier, with a vessel or object used in research. RFIDs are typically associated with a biological research reagent or product by either attaching an RFID tag to a surface of a vessel or an object that contains, holds, or supports the research reagent, or is itself part of the research reagent, or embedding an RFID tag within the vessel or object. Virtually any technology available for associating an RFID tag with a vessel or other object, such as a bottle, tube, slide, gel, a bead, a particle, etc. can be used with the present invention.

For example, a “smart label” can be used, which includes an RFID inlay (a chip and antenna combination, i.e. an RFID tag) contained within an adhesive label. A smart label can include a tag embedded in label material that is printed with human-readable text, graphics, and bar codes. Smart label printers encode the RFID chip inside of the label material and can print text, bar codes, and graphics on the outside. In certain aspects, provided herein is a biological product that is associated with a label that includes an RFID tag, and optionally human readable information, such as printed text, and optionally a barcode.

In certain illustrative examples, the RFID is embedded within packaging or a vessel, support, or other structure that holds a research reagent. By embedding the RF tag in the vessel or other object used to contain or immobilize a research reagent, the RF tag becomes permanently associated with the vessel or object. In this case, it is not necessary to apply an RFID tag during manufacture of the reagent.

In a related embodiment, the biological research product itself can be, for example, a plastic structure or vessel, such as a plastic bottle, plate, cassette, or tube. The RFID tag can be embedded within the plastic vessel during an injection molding process.

In one illustrative aspect, an RFID tag is embedded within a plastic vessel or the plastic lid of a vessel of any composition that will contain a biological research reagent. During the bottle or lid molding process an RFID tag is placed at the bottom of the mold before the plastic is injected into the mold. This results in the RFID tag being trapped and molded into the plastic. In another illustrative aspect, the research product includes a plastic vessel and a plastic object including an RFID tag, wherein the plastic object is secured within a cavity of the plastic vessel. Accordingly, if the vessel is a tube, for example, a secondary RFID tag plastic button can be molded to fit in to the bottom of the tube. The tube's bottom or a lid of a container can have a cavity that will accommodate the shape and size of the RFID tag button. The RFID tag button can be pressed into the bottom of the tube at a manufacturer and delivered assembled to a customer.

Accordingly, provided herein is a method for making a vessel comprising a radio frequency identifier (RFID), that includes depressing an object that includes an RFID into a cavity of a vessel, or inserting an RFID tag to a vessel mold, wherein the vessel comprises a biological research product label. The vessel is a plastic vessel can be, for example, a plastic tube or other plastic object such as a cylinder, for example with an end having a diameter of less than 1 centimeter. The object that includes the RFT can be a button.

RFID tags can also be embedded in plastic or paper-based structures, for example, the wall of a gel cassette or vial, or the rim of a multiwell plate, or a collar that fits around a tube, vial, or column.

In some cases, the research product to be identified is itself a structure such as but not limited to: a gel, a gel strip, a filter or membrane, a plate, a chip, or an array. In these cases, the RFID tag is attached to or embedded in the object it identifies.

In other exemplary embodiments, the research reagent RFID tag can encode information on the sample type tested or processed using the reagent. Preferably, in these embodiments, the RFID tag associated with the research reagent is writable, and sample information such as the identity of a sample can be written to the tag by the user. The research reagent RFID tag can alternatively or in addition have information on the protocol to be performed using the reagent. In some preferred embodiments, protocol information can be written to the RFID tag by the researcher. Results of a protocol can optionally also be written to the tag by the user.

In some embodiments, the RFID tag has memory that is writable and erasable. In other embodiments, the RFID tag has information that is “locked” in memory storage and has additional memory capacity that is writable and erasable.

The invention therefore includes: a biological research product that includes a writable RFID tag that includes information that includes one or more of: 1) the type, quantity, concentration, or identity of one or more components of the biological research reagent (reagent identity information); 2) the type, quantity, concentration, or identity of a sample that is to be used in a procedure with the biological research reagent (sample identity information); or 3) one or more procedures to be performed on a sample with the biological research reagent. In preferred embodiments, the writable RFID tag on the biological research reagent has sufficient memory space to accommodate additional information added during the course of the use of the research reagent by the user.

In some exemplary embodiments, a research reagent with an RFID tag is a research product such as a gel or gel cassette, a filter or membrane, an array, a chip, or a plate, such as but not limited to a multiwell plate; in which one or more assays, separations, syntheses, processing steps, or reactions can be performed in or on the research product, and information about a sample applied to the research product can be written to the RFID tag attached to or embedded in the research product. Information about the procedures performed on a sample can also be written to the RFID tag associated with the research product. The procedure-based information written to the RFID tag can be information on the parameters of the procedure, for example, electrophoresis conditions, incubation time, incubation temperature, etc. Alternatively or in addition, result-based information can be written to the tag, such as, for example, the molecular weights of bands detected, the intensity of fluorescence from a detection assay, etc.

Thus, another aspect of the invention is a biological research product that includes a writable RFID tag that includes information on the identity of one or more components of the biological research reagent (reagent identity information). The writable RFID tag on the biological research reagent has memory space that can accommodate additional information added during the course of the use of the research reagent by the user. For example, the user can write to the tag information on the type, quantity, concentration, or identity of a sample that is to be used in a procedure with the biological research reagent (sample identity information); or can write to the tag one or more procedures performed or to be performed on a sample with the biological research reagent.

The information encoded on the RFID tag can be read and transmitted to a processing unit where the information can be stored.

Readers typically include one or more antennas for sending and receiving signals to and from tags and a processor for decoding received signals and data. Collected data is then passed through normal interfaces (such as a cable or wireless LAN) to a host computer system.

Based on the amount of memory in a tag and how it is designed, readers used in certain aspects of the present invention can also program new data into tags. Readers used in methods and systems herein, typically operate in accordance with local (national) RF emission regulations; tags and readers used in the methods, systems and products herein typically conform with particular specifications and standards in order for them to communicate in a well defined manner. In certain aspects, the reader is a “Frequency agile” reader capable of recognizing multiple frequencies. In other aspects, multiple readers are utilized that support different frequencies at each read point to ensure all tags are processed.

Application requirements determine the frequency, memory, and performance requirements for the tags to be used. Other considerations include whether the tag will be used globally and what interoperability standards (if any) the tag must meet.

The products, systems, reagents and methods provided herein can use, for example, passive RFID tags with the following characteristics:
Low Frequency RFID systems operating at about 125 kHz with a typical maximum read range of up to 20 inches (508 mm).
High Frequency RFID systems operating at 13.56 MHz with a typical maximum read range of up to 3 feet (1 meter).
Ultra-High Frequency RFID system operating at multiple frequencies, including 868 MHz (in Europe), a band centered at 915 MHz, and 2.45 GHz (microwave). Read range is typically 3 to 10 feet (1 to 3 meters), but systems operating in the 915 MHz band may achieve read ranges of 20 feet (6 meters) or more.

In certain aspects, the present invention utilizes a low-frequency radio frequency identification systems (30 KHz to 500 KHz) having short transmission ranges (generally less than six feet). In other aspects, the invention utilizes high-frequency RFID systems (850 MHz to 950 MHz and 2.4 GHz to 2.5 GHz), which offer longer transmission ranges (more than 90 feet). A skilled artisan can determine an appropriate transmission range for an RFID-labeled research product provided herein depending on the particular requirements for the product and methods that utilize the product.

Typically, tags and read/write devices used in methods herein share more than the same frequency to communicate. Compatible encryption and decoding algorithms, data content and format, interface protocols, and other technical specifications are typically also compatible between tags and read/write devices. In certain embodiments, the tags include information that is standardized so that it can be read across organizations.

Certain aspects of the invention include systems that include a biological research apparatus and a reader. RF Readers for use with the present invention can be integrated into handheld terminals; fixed and positioned at strategic points, such as a laboratory entrance, or biological reagent manufacturer and/or distributor assembly line; or integrated into laboratory equipment such as gel electrophoresis boxes, thermocyclers, gel scanners, imaging devices, etc.

In fact, in another embodiment, the present invention provides information stored in computer readable form that identifies an object of biological research, and an identifying symbol(s) that identifies the type of object.

The data transmitted from the tag to the reader antenna can be used to notify a programmable logic controller that an action should occur. For example, in the present invention, the action could be the creation of a digital image of an identifier (for example, a label) that is added to an image of a stained gel.

### Gels

In another aspect, which itself forms another embodiment of the invention, the biological research product is a gel or includes a gel localized on the surface of the object. For example, the gel can be made of any substance known in the art of gels, especially electrophoretic gels for analyzing biomolecules. The gel can include, for example, agarose or acrylamide, or a combination thereof. Furthermore, the gel can be a gel strip or a series of gel strips, or a slab gel, such as a pre-cast slab gel. In one aspect, provided herein is a precast gel that includes a RFID tag embedded within the gel, embedded within a gel cassette that supports or contains the gel, or attached to the surface of the gel or the gel cassette. In certain illustrative aspects, the gel is an e- gel, or an e-PAGE gel, such as an e-PAGE 96 or e-PAGE 48 gel, or a NU-PAGE gel (Invitrogen, Carlsbad, Calif.).

Tags embedded in gels can preferably but optionally be low frequency RFID tags that are read at relatively close proximity, as low frequency chips are less likely to have problems of interference in proximity to liquids. In some preferred embodiments, gels having embedded RFID tags that are used for the separation of biomolecules such as nucleic acids and proteins are less than 5 mm thick, preferably less than 3 mm thick, and in some preferred embodiments can be less than 2 mm thick. Preferably, for gels that are run in the presence of a buffer system that surrounds at least a portion of the gel, a gel-embedded RFID tag is read before the gel is positioned in an electrophoresis apparatus that includes buffer, or after the gel has been removed from the apparatus.

In one illustrative example, provided herein is a method for labeling a gel, wherein information is written to an RFID tag that is associated with the gel. For example, a date and identity of samples in wells on the gel can be written on the RFID tag. The information remains with the gel during storage, optionally in addition to information provided by a manufacturer regarding gel type, serial number, etc. The information can identify each gel and each gel type provided to a customer by a provider. a tag that can be inserted in to the gel cassette before the gel polymerizes. A handheld RFID reader device, such as a reader attached to a portable computer device such as a PDA can be used. Thus the invention allows information to be easily read by a user, allows a user to distinguish all gels (e.g., by a unique number for every single gel manufactured by a provider), and allows a physical tag to remain associated with a gel.

Provided herein in another embodiment, is a gel scanner system that includes a computer in communication with a gel scanner, and an RFID reader in communication with the computer and/or the gel scanner. Gels scanned using the system typically have an associated RFID tag. The system allows information to be read and written between the components of the system, to create an extremely powerful system for managing gels and results thereof. The system can be used by a laboratory to manage storage of gels after performing an experiment using the gels. For this task, a single reader or a series of readers at various locations in the lab can be used. A technician can periodically scan various areas of a laboratory with a reader to identify locations of all the gels of the lab, then, if necessary connect the reader to a computer system such that a database that includes an image of a gel and other info regarding a gel, is updated with the location of a gel within the lab.

Furthermore, the information on the RFID, regarding, for example, gel type, serial number, etc., can be read by the RFID reader and communicated via the RFID reader to the computer where it can be combined with an image from the gel scanner on the computer, such that information regarding the gel, from the RFID tag associated with the gel is added digitally to the scanned image of a gel. This provides the functionality of a non-digital marking system, but in a more legible, more convenient, and more permanent manner, and with additional functionality. The gel scanner can also identify molecular weights, for example, of protein on lanes on the gel and write the molecular weight information to the reader, so that the reader includes information regarding date of experiment, samples in wells, and results of experiments performed using the gel.

In another embodiment, the invention provides a gel electrophoresis apparatus that is associated with an RFID reader that communicates with a computer system such that when a gel is placed in the electrophoresis system, the reader reads an identifier on the gel and communicates this to the computer system which communicates via the Internet or other wide area network to a gel manufacturer computer server. The server identifies relevant information regarding the gel, for example an expiration date, relevant QC data, the latest version of a product manual from the gel, whether the gel is of a lot that has been identified as being unsuitable for some reason, etc. A computer system that can include a computer display on or near the gel box, then relays information to a gel user. For example, if a gel is past its expiration date, the computer system will automatically notify the customer on the LCD screen with a signal such as “WARNING GEL PAST EXPIRATION!” This can be performed without customer intervention. In certain aspects, when an expired reagent is detected, a provider is notified and automatically sends a request to a customer of a lab that contains the expired reagent, inquiring as to whether a new reagent should be sent, or the system can automatically send new, unexpired reagents to a customer.

In another example, an RFID reader is connected to a low intensity LED readout that is placed on or near a gel and photographed in the same image as the gel. Alternatively, for a transmitted light image of the gel, the readout can be a miniaturized version of a projection LCD display where the numbers corresponding to the gel are darker than the transparent LCD.

Biological Arrays

In another aspect that itself forms a separate embodiment of the invention, provided herein is an RFID-labeled biological research product that includes an array of biomolecules localized on the surface of an object that includes the array. In one example, the array is a membrane onto which individual biomolecules, such as antibodies, proteins, or nucleic acids, are covalently attached at specific locations on the array. In another example, the array can be a glass slide having attached biomolecules. The array can be a microarray, for example of 100 mm or less in an single dimension (for example, having dimensions of 25 mm×75 mm or smaller) and can optionally be part of a research product that includes channels and microfluidics.

In some preferred examples the array is a high-density array that includes biomolecules immobilized on the surface of a substrate such as glass at a concentration of greater than 100, 200, 250, 500, 1000, 2500, 5000, or 10,000 biomolecules/cm².

Arrays can include RFID tags attached to a surface of the array or embedded in, for example a polymeric or fiber-based array. The RFID tag associated with the array includes an identifier that can be read to provide the user with information on the type of array—for example, by reading the tag the user can immediately know the types of molecules on the array (antibodies for proteins of a particular class, for example). In preferred embodiments, the user can write sample information to the tag. The sample information and array identity information can be read when the chip is scanned after a hybridization assay to detect positively interacting antibodies (for example, by detecting fluorescence). Sample and array information can be integrated with a digital image of the scanned array by a processing unit and used to analyze the hybridization results. No data by the user input is required for this analysis.

Functional Chips

Functional chips includes chips on which cell and biomolecule separations, cell an biomolecule capture, binding detection, functional assays, biochemical reactions, and biochemical synthesis can be performed. Often functional chips are used in workflows in which one or more components of a sample is separated or concentrated, and then further analyzed, for example, to detect a particular analyte or biomolecule, in an activity assay, or to identify a particular nucleic acid or protein sequence. Functional chips can include channels, wells, electrodes for cell and biomolecule separation, Peltier elements for heating, electromagnetic elements for particle capture, acoustic elements for mixing, sensors, etc.

The present invention includes functional chips having associated RFID tags. The RFID tags can be embedded in the surface of a chip, or attached to the chip. Preferably, the RFID tag that is on or in a functional chip encodes a product identifier that provides non-erasable information on the chip (such as a part number), and optionally, its function. The RFID tag associated with the chip preferably is also writable. Information on the sample to be applied to the chip, and, optionally, experimental parameters can be written to the tag. In preferred embodiments, one or more experimental results is written to the RFID tag on the functional chip. The information can be used to determine a downstream procedure using the same chip. For example, an assay may be repeated, or a second assay may be performed on the sample on the chip.

Systems for Sample Analysis that Include RFID Readers

Systems for performing at least one a biological research function, in which the system includes at least one RFID tag reader and a processor for converting the information read by the reader into stored information are another feature of the invention. The systems use at least one research reagent that includes an associated RFID tag. The RFID tag reader of the system can receive information stored on the RFID tag of the one or more research reagents. The information stored on an RFID tag and received by the system reader is information concerning the research reagent, information concerning one or more samples associated with the research reagent; and/or information concerning one or more biological research functions that has been performed on a sample associated with the research reagent or is to be performed on a sample associated with the research reagent.

The biological research function can be, as nonlimiting examples, an assay, reaction, separation, biochemical synthesis, or sample processing step. In some embodiments, the system can perform more than one type of biological research function on a sample. The sample can be any type of sample, and is preferably an environmental or biological research sample. A biological research sample can comprise one or more biomolecules that are separated, detected, or assayed by the system. In some embodiments, a sample is not a clinical sample collected from a human subject.

In some exemplary embodiments, a research reagent with an RFID tag used in these methods is a research product such as a tube or vial, a gel or gel cassette, a filter or membrane, an array, a chip, or a plate; in which one or more assays, separations, syntheses, processing steps, or reactions can be performed in or on the research product. The processing unit of the system can correlate information from the RFID tag associated with a research product with results of an assay, reaction, biochemical synthesis, or processing step performed on a sample provided in or on the biological research product.

The invention includes methods of using a biological research reagent that includes an RFID tag in an experimental protocol, assay, or procedure, or in sample processing. The RFID tag includes one or both of information on the type, identity of the reagent; the type or identity of a sample to be used in a procedure with the biological reagent; or one or more procedures to be performed on a sample using the biological research reagent. In some preferred embodiments, the method includes reading the information on the RFID tag, in which the information provided on the RFID tag controls or directs at least one assay step, separation step, reaction, synthesis, or processing step that is performed on a sample using the biological research reagent.

The present invention provides systems for analyzing samples using RFID-tagged biological research reagents, where a system comprises at least one RFID tag reader; a powered device for performing a function selected from the group consisting of an assay, detection, reaction, biochemical synthesis, and sample processing step on a sample; and a processing unit for storing information from the reader. The processing unit is operatively linked to the RFID tag reader and to the powered device for performing the biological research procedure, and the processing unit can store the information read by the reader and link information read by the reader to information on the parameters or results of the performed procedure.

The system is used with one or more RFID-tagged vessels, supports, or structures that can hold a sample to be analyzed. In some preferred embodiments, a procedure is performed by the system on the sample while it is in or on the tagged research product, for example, a tagged gel, separation strip, array, plate, or chip.

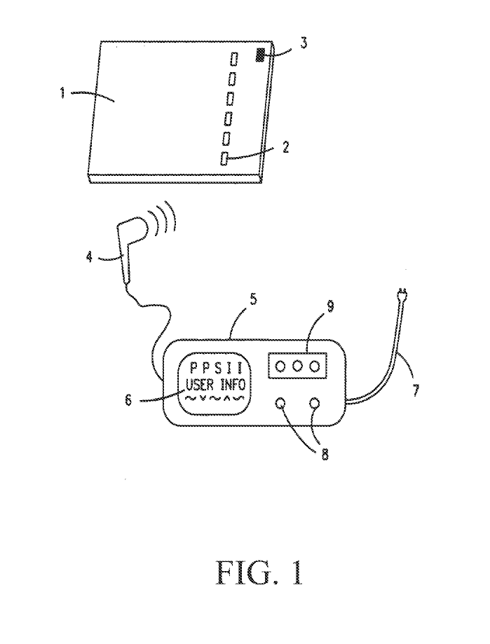

In some embodiments, a powered device of the system is a power supply suitable for electrophoresis applications, such as but not limited to gel electrophoresis, isoelectric focusing, or solution isoelectric fractionating. FIG. 1 depicts an RFID system that is used to read information encoded on an RFID tag (3) that is embedded in a gel (1) having wells (2) for loading samples. In this case, a handheld RFID reader (4) is connected to a power supply (5) that can be connected to a standard voltage outlet by a power cord (7) and to leads that can connect to an electrophoresis apparatus through lead connectors (8). The power supply/reader system includes a voltage/current readout panel (9) and a display screen (6) that can display pertinent information received by the reader from the RFID tag (3) embedded in a gel, or linked to information provided on the tag, such as, for example, preferred run times and/or voltage or current setting, preferred buffers, information on the concentration of the gel and composition of the gel buffer.

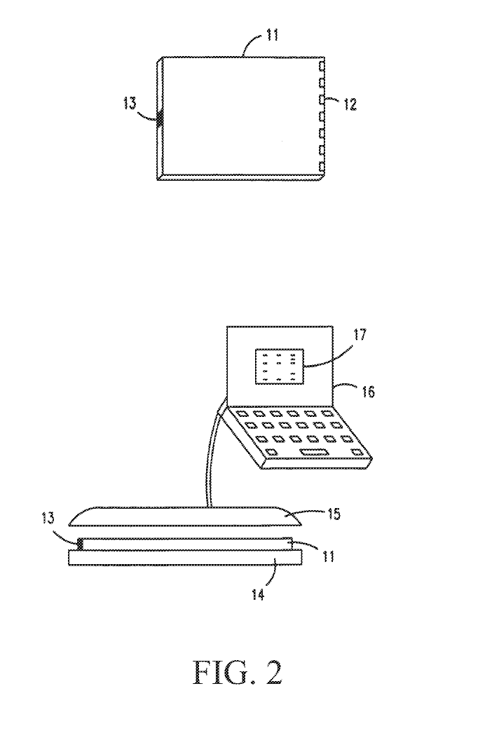

In some embodiments, a powered device of the system is a scanner that detects fluorescence, radioactive emissions, or optical absorption. A scanner can be used to read plates, membranes or filters, plates, arrays, or gels. FIG. 2 depicts an RFID system for bioresearch that includes a gel or filter optical scanner (15) linked to a personal computer (16) that can be used to scan a gel within cassette (11) that has an attached RFID tag (13). The cassette which comprises transparent plastic (11) can be placed on a visible light source (14) (such as the Safe Imager (Invitrogen)) and stained molecules within the gel can be detected with the scanner (15) that also includes a reader for reading information encoded in the RFID tag (13) on the cassette (11). Information about the gel samples loaded into wells (12) and electrophoresed on the gel in the cassette (11) that is received by the reader, as well as a digitally encoded image of the scanned gel, can be communicated to a personal computer (16), where an image of the scanned gel can be displayed (17) and stored

In some embodiments, a powered device of the system comprises a heating element. This allows the system to be used for reaction incubations and/or denaturing steps. The powered device can be a thermocycler.

In some embodiments, the powered device of the system comprises a power supply and circuitry to provide a signal source to electrode configurations, electromagnetic elements, Peltier elements, acoustic elements, or microfluidic devices for cell or biomolecule separation. The separations or reaction performed by the system can be performed on functional chips, for example.

Preferably, an RFID biological research system's processing unit can direct at least one procedure using information read from an RFID tagged biological reagent using the reader. In preferred embodiments, the processing unit can integrate and store sample information read from an RFID-tagged biological reagent with information on the parameters of a procedure performed by the system. Such parameters can include temperature, incubation time, reagents used, etc.

In some preferred embodiments, the system's processing unit can integrate and store sample information read from an RFID-tagged biological reagent and correlate the information received from the tag with information on the results of a procedure performed by the system.

In preferred embodiments, the system also has an RFID writer that can enter information on the parameters or results of at least one procedure performed by the system on an RFID tag associated with a biological research product that holds, contains, or supports a sample.

In further embodiments, the system's processing unit can direct at least one additional procedure on a sample based on the results of a first procedure performed on the sample by the system. This provides an intelligent automated system for sample processing and analysis. For example, a cell separation on a functional chip can result in the selective retention of, for example, malignant cells. Detection of the cells using a labeled antibody can be recorded by the processing unit, and the positive detection result can be written to the RFID tag on the chip. The tagged chip can be directed to a cell lysis/PCR workflow for an assay to detect an isoform of a gene of interest expressed in the detected cell type.

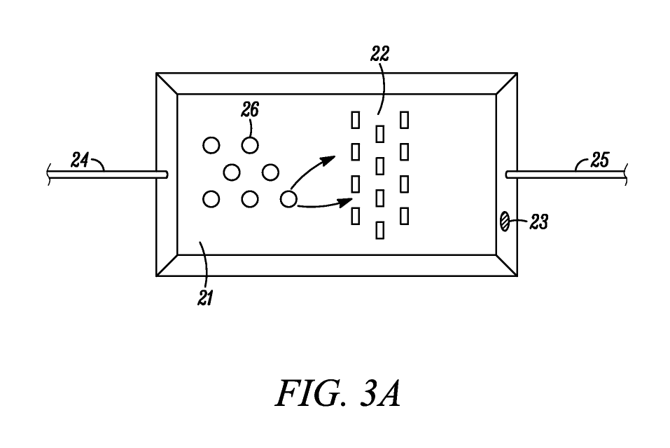

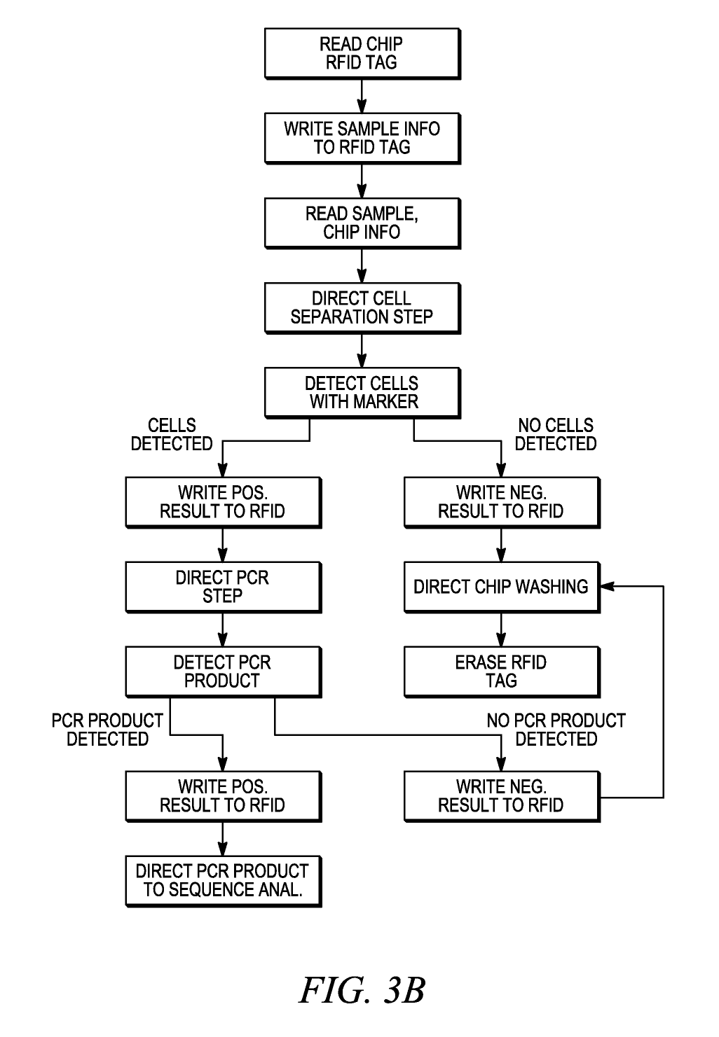

In some preferred embodiments, the processing unit is operably linked to a personal computer, and software application can be used to analyze, graph, or image the data generated by the system. The processing unit can also be operably linked to a database. The database can be a gene or protein sequence database, a molecular structure database, a technical services database, a scientific literature database, a cell line database, etc. INVIT1380-1 (24) and outflow conduit (25) that can be opened an closed for control of sample loading and washes, and an assay area (22) for the capture of sample components such as cells (26) that can be introduced onto the chip surface. The biochip (21) has an attached RFID tag (23) that can transmit information to a reader associated with the biochip analysis system. FIG. 3B depicts a workflow of processes that can be performed on the functional biochip (21). Detection devices such as optical scanners can determine the results of capture assays and PCR, and sample information and separation, assay, or biochemical synthesis results can be read from and written to the RFID tag (23) attached to the biochip (21). The information encoded on the RFID tag can be used, through interfacing software, to direct steps in the analysis of a sample, as indicated in the workflow diagram.

Methods for Performing Experiments that Include Reading and/or Writing Information to a Radio Frequency Identifier (RFID) Associated with a Biological Product

In another embodiment, provided herein is a method for performing an experiment, that includes reading and writing information to a radio frequency identifier (RFID) tag associated with a vessel containing a biological research product or an object associated with a biological research product. The method includes reading and writing information to a radio frequency identifier (RFID) tag associated with an array or biomolecules. For example, the method can include reading information regarding the identity of a tagged research product to be used in the experimental protocol, and writing information regarding an identity of a sample applied to the tag on the research product. In preferred embodiments, the method is a method that involves a biological sample, but is not a method used for clinical purposes. Therefore, the method is preferably other than a method that directly relates to diagnosis, monitoring, prognosis, detection, or treatment of a medical condition.

In another aspect, the method includes reading and writing information to an RFID that is embedded within a substrate of an array or to an RFID that is attached to the surface of a substrate of the array. In another aspect, the method includes reading and writing information to an RFID regarding a sample that is applied to the array.

In one aspect, the method includes reading and writing information to a radio frequency identifier (RFID) associated with a biomolecule separation gel or gel cassette, such as a protein separation gel or a nucleic acid separation gel or cassette, or associated with a filter or membrane, such as a filter used in a blotting procedure. The method can include, for example, writing information regarding an identify of a sample loaded into a well of the biomolecule separation gel, or information regarding the identify of a series of samples loaded into a series of wells of the biomolecule separation gel.

In methods provided herein, information can be read and written one or more times and at one or more, for example all steps of the process. For example, information can be read from and/or written to the RFID tag at least 2 times, for example, 2, 3, 4, 5, 6, 7, 8, 9, or 10 times during the method.

In another aspect, information on the RFID tag is used to affect a step of a chemical or biological research method, or information is written to the RFID tag during a chemical or biological research method. The method includes reading information from the RFID before performing a step of the method. The biological research reagent can be provided in a tube or vial that is RFID-tagged, or the biological research reagent can be a functional chip on which one or more research procedures is performed. The reading or writing can affect the performance of a subsequent step. For example, a step can be eliminated or included depending on information that is encoded by the RFID tag associated with the biological research reagent and read by an RFID reader that communicates with an instrument or a mechanical sample processing system such as a conveyer belt and/or a switch or router. Furthermore, a setting used to perform a step, such as time, temperature, voltage, current, etc. can be set or varied depending on information that is encoded by the RFID tag associated with the biological reagent and read by an RFID reader that communicates with an instrument.

In certain methods, a plurality of RFID readers can be placed in any or all storage areas of a lab such that they read the RFID tags on all biological research products within the lab. The RFID readers are connected to a lab computer system. The readers regularly (e.g. daily) read information from RFID tags, or the tags regularly transmit information wherein the tags are associated, such as being embedded or affixed, to a biological research product, such as a gel, array, reagent, etc. within the lab such that if any item is past its expiration date, a warning instantly comes up on a lab computer, identifying the name and location of the product that is expired. Therefore, provided herein is a method for identifying expired research reagents.

In certain aspects, the RFID-labeled biological research reagent product is stocked within a supply center. A supply center is a storage location at a customer facility, where a supplier stocks supplies such that the supplies are shipped and stored at the storage facility before they are ordered by the customer. In related aspects, provided herein is a Supply Center that includes on or more RFID readers. For example, a plurality of antennae can be included at various locations on the Supply Center to transmit information about each of the locations (e.g. shelves) to a reader.

In another aspect, information is read and/or written to an RFID tag during a nucleic acid amplification protocol. For example, a thermocycler can include an RFID writer and read and write the number of cycles performed to an RFID tag on a tube being cycled in the thermocycler.

Methods provided herein can be automated methods, such that reading and writing information on an RFID tag is used to affect an automated step during a biological research reaction. RFID tag information can, for example, be used by a robotic system to direct and/or affect steps performed by the system.

In certain aspects, the methods can include a temperature sensor that can be integrated with the RFID tag. The RFID tag can be interrogated by the reader to determine temperature of the biological research reagent.

In certain methods, such as those involving plates or microarrays, an RFID tag associated with the plate or microarray is read and/or written during or after a research procedure, thereby allowing a user to label the plate or array, for example, with information regarding an experiment performed using the plate or array (date, assay type, sample applied, probe etc.). Furthermore, as indicated above, information on an RFID tag can be used to affect an automated process. For example, a user can label a plurality of plates, such as, for example 100 plates, with different information regarding the sample that is to be applied to various wells in the plate, and a robotic system can read the RFID to determine which reagents are dispensed in which wells on which plates.

Biological Defense

In another embodiment, provided herein is a method for tracking a biological sample for an agent of biological warfare, that includes reading and/or writing information to a radio frequency identifier (RFID) associated with a biological sample collection vessel, container, plate, or filter.

### Methods Involving Determine an RFID Location

The invention further includes methods which combine radio frequency identifiers (RFIDs) with one or more of the following: (a) bar codes, (b) global positioning systems, and (c) computer based tracking.

The invention also includes methods for identifying the location of one or more RFIDs. The location of an RFID in an area may be determined by any number of methods. One example of such a method is the signal strength of the RFID when read at one or more locations. As an example, differences in signal strength from an RFID, as detected by three separate receivers may be used to determine the location of the RFID by triangulation.

Further, once the location of one or more RFIDs are determined, either the movement of an individual RFID or the collective movements of multiple RFIDs may be monitored and/or recorded. One application of recording such movements is in athletic events. During many athletic events (e.g., hockey, football, soccer, baseball, etc.), the positions and coordinated motions of the participants is critical to the success of teams. Also, in many instances, after an event, the coordinated motion, or lack thereof, is reviewed as a teaching tool for the participant. Thus, the invention includes the use of RFIDs, and systems which determine the locations of the RFIDs, to track the movement of one or more individuals in athletic events. In many instances, systems which determine the locations of the RFIDs will contain a computer for data analysis and/or recording.

Participants in athletic events may each have one or more RFID on their person. It may be advantageous for participants to wear more than one RFID when, for example, one of the RFIDs is a passive RFID and another is an active RFID.

Methods of the invention also induce the tracking of items (e.g., people, baggage, etc.) in areas where there is a need to maintain security (e.g., an airport, on airplanes, seaports, places where public events are held, etc.). Methods of the invention are particularly useful for ensuring that individuals are on airplanes along with their baggage. Thus, the invention includes methods for determining whether individuals who have checked baggage on a flight are present on the airplane at the time of departure. These methods include associating an RFID with individuals who board the plane and determining whether the assigned RFIDs are located on the airplane at the time of departure.

Methods of the invention also include the identification of individuals in an airport, as well as determining the location of those individuals in the airport.

Methods for associating RFIDs with individuals include embedding RFIDs under skin and in identification documents (e.g., passports, driver's licenses, etc.).

The invention also include methods for keeping track of the location of animals (e.g., livestock) which may have one or more infectious diseases. As an example, all or substantially all of the animals in a group (e.g., animals present on a ranch) may each have an RFID associated with them. Further, the movement of these animals may be determined and/or recorded using methods of the invention. If one animal in the group is found to have a disease (e.g., mad cow disease, Rous sarcoma virus, etc.), then it will be possible to identify other animals that had contact with the sick animal. Thus, allowing for, for example, quarantine of the additional animals that may have contracted the disease through close association.

### Kits, Matched Reagents, and Multiple Reagent Workflows

The invention provides kits for biological research that include reagents that are labeled with RFID tags and/or wherein kit packaging is labeled with RFID tags.

In one embodiment, the invention provides kits that include two or more RFID-tagged biological research reagents. The tagged research reagents can be used in a common protocol—as, for example, an enzyme and cofactor, or labeling reagent and labeling reaction buffer. The RFID tags on the reagents can provide information on the expiration dates, can link to technical information on a website, etc.

In some embodiments, many reagents may be provided in a kit, only some of which should be used together in a particular reaction or procedure. For example, multiple primers may be provided, only two of which are needed for a particular application. RFID tags associated with these reagent can assist a technician in selecting the appropriate reagents.

Yet another aspect of the invention is a set of two or more biological research reagents that can be used in a common workflow, in which each of the two or more reagents is RFIDtagged. In general, the matched reagents are not used simultaneously, but rather in different experiments which may or may not necessarily occur in a sequential order.

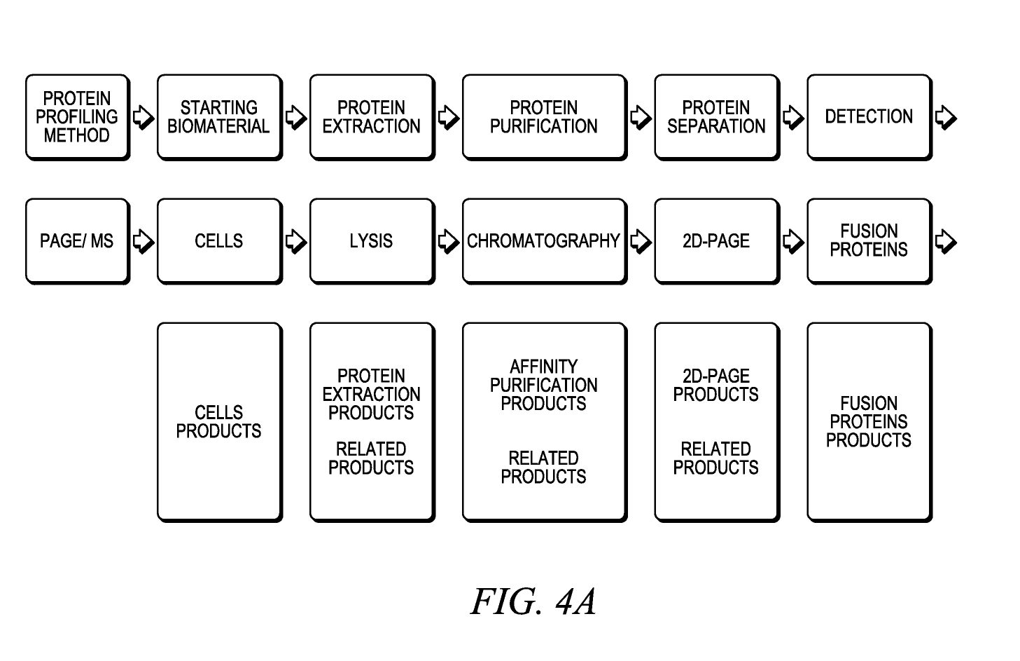

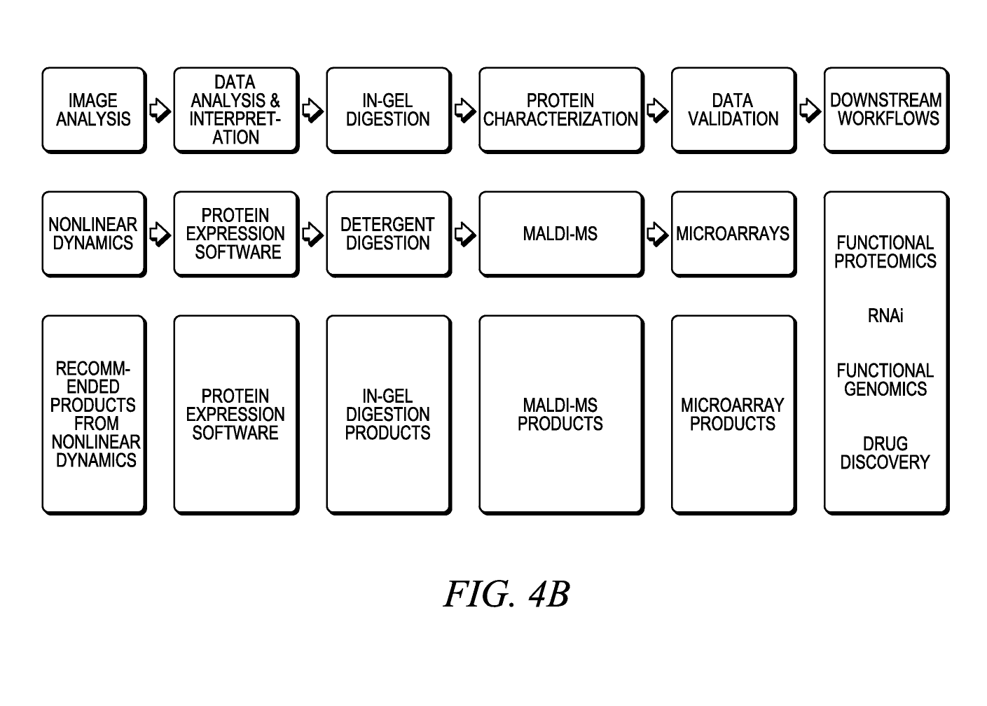

Provided herein therefore is a plurality of reagents that are used in a common biological research workflow, wherein at least 1, 2, 3, 4, 5, 6, 7, 8 ,9, 10, 15, 20 or all of the reagents and/or instruments in the workflow are associated with a radio frequency identifier (RFID), an RFID writer, and/or an RFID reader. In certain examples, the workflow is a gene or protein expression profiling workflow, an RNAi analysis workflow, or a protein-protein interactions workflow. For example, the biological pathway can be a protein expression profiling workflow (FIG. 4) within a functional proteomics discipline. The RFID tags, readers, and writers communicate with each other and preferably a communication system that can include a laboratory information management system (LIMS) and optionally a computer system of a biological research reagent provider, through a wide area network, to improve the efficiency, accuracy, and success of biological research.

The present invention also includes a biological research system, comprising at least two instruments or reagents that are used in a first biological workflow, in which at least two of the instruments and/or reagents comprise a radio-frequency identifier (RFID) tag, an RFID reader or an RFID writer. The biological research system can include reagents used in a is gene or protein expression profiling workflow, RNAi analysis workflow, or protein-protein interaction analysis workflow. The workflow can use biological research products such as a gel or microarray.

The biological research system can be part of a laboratory information management system, and can have protocols and controls linked to a common processor. Information can be communicated between at least two instruments and/or reagents that are used in the workflow, and the information can be stored on radio frequency identifier (RFID) tags, RFID readers, and/or RFID writers associated with at least two of the instruments and/or reagents used in the workflow.

The invention further provides a set of matched biological reagents associated with a target biomolecule, a biological research kit, or a target biomolecular workflow, in which multiple vessels, each containing a member of the set, are each associated with a radio-frequency identifier (RFID) tag.

The invention further provides a collection of matched biological reagent sets, each of which is used in a separate workflow, in which the target biomolecule, the biological research kit, or the target biomolecular workflow associated with each set is identified on information stored on the RFID tags associated with all of the vessels of the set. A collection of claim 83, can include two or more sets, each of which is associated with a different target biomolecule. For example, each different target biomolecule associated with a different set can be a different gene, or an open reading frame of a different gene, such as, for example, a mammalian gene. For example, the gene associated with a set of matched reagents can be a human gene.

In another example, the target biomolecule that different sets of matched reagents are associated with can be an enzyme and a matched reagent set can comprises buffers in which the enzyme has enzymatic activity.

The invention also provides a method for determining whether two biological reagent vessels comprise matched reagents, comprising: reading reagent identification information on a radio frequency identifier (RFID) tag associated with each of the two biological reagent vessels; and using the information read from the RFID tag to determine whether the two biological reagent vessels contain reagents that are associated with a target biomolecule or a target biomolecular workflow.

The following examples are intended to illustrate but not limit the invention.

### EXAMPLE 1

### RFIDs Associated with Supply Centers

This example illustrates the use of RFID in a supply center.

The present Example discusses the use of RFID technology associated with biological research reagents in the context of a biological reagent/product supply center. Billy has a biological reagent/product supply center inside his company (the BR Supply Center). Vendors stock these centers with products Billy needs on a day-to-day basis. This removes the process of Billy having to place an order, and wait for it to be delivered. One of the biggest problems is that under the standard procedure, Billy is “not charged for this stocked product until he checks it out from the BR Supply Center”. Under the current system, Billy walks into the BR Supply Center, finds what he is looking for and checks it out by writing down his department code, part number, and quantity.

The procedure has several pitfalls. First, Billy frequents the BR Supply Center three to four times a day; he occasionally becomes lazy and does not check out the product correctly or not at all. Often he thinks he will check it out when he comes back for the fifth time, but it doesn't always happen that way. This creates a billing nightmare and results in a loss of money for the vendor, as “the Customer is always right”. In addition, when Billy comes the next day and realizes that he took the wrong product, it becomes a nuisance to return the item. If a new check out sheet has been issued, Billy is unable to simply cross out the mistake. He needs to follow some other procedure to ensure he does not get charged; or he might just replace the item and take the correct one. He does this with out checking it out (“hey the prices are about the same”).

Another loss in efficiency results from the fact that Billy is not the one stocking the shelves, rather it is the vendor who does the stocking. The vendor requests X amount of a product to be sent to Billy's company, and the amount is based on the historical consumption of goods. This data is collected from the sheet that Billy fills out when taking products out of the Supply Center, which of course can have errors. Once the product has been shipped and delivered to Billy's company, the vendor spends hours checking in the products and stocking the shelves. The vendor also checks to see if the BR Supply Center is getting low on any products. Once done with that, the vendor reconciles Billy's transactions, and bills the company. As you can see, there is a lot that goes on in a supply center

Now lets see what happens when we put an RFID tag on the product and the BR Supply Center is made “smart” by including RFID tags associated with the biological research reagents and products sold at the Supply Center. Billy is going to walk into the Biological Reagent/Product Supply Center, take what he needs, and walk out. The BR Supply Center can print out an accurate receipt based on the goods taken by Billy, or e-mail Billy a receipt.

As for the vendor reordering and restocking, it is all integrated. When Billy walks out of the Smart BR Supply Center, he leaves with nine boxes of product Y, leaving two remaining on the shelf The Smart Supply Center recognizes this, calls up the main distribution center, and requests more product Y. The Smart Supply center can “see” every item on its shelves by monitoring the RFID tags associated with the products. In effect it can perform its own physical inventory in an automated fashion. Further, since each item is individually identifiable, if Billy realizes he took the wrong product and needs to return it, all he needs to do is walk back to the Smart Supply Center, and place the produce back on the shelf. That's all it takes. Historical trending of product purchases can also be performed in a more accurate way. In addition, inventory in supply centers can be smaller due to real time inventory data, which keeps costs down.

There are further benefits at the customer level. Currently when Billy receives his order he is on his own. If he needs to get product information like a manual, material safety data sheet, or certificate of analysis, his options are either search the web site or make a call to the supplier's technical service department. If Billy contacts Technical Services and requests information, he will either receive it by e-mailed, fax, or standard mail. All this information is available on the web site, but Billy may not have access, or may be too busy to search for it. This could be simplified by using RFID tags.

If the product was made smart by installing RFID tags and readers that interface with Billy's computers, Billy could find what he is looking for with a few clicks of a mouse. This is made possible by a piece of hardware that attaches directly to Billy's computer that reads the RFID tag on a product and. enables Billy's purchased items to communicate directly with his computer. The computer receives information specifically about Billy's items. This information contains a part number, lot number, expiration date, even information about Billy's purchase like the date the item was ordered, ship date, and Purchase order number. This information is relayed to Billy's computer where it automatically links to the supplier's web site and retrieves a customized page with all information available for that specific item, including a user manual, material safety data sheet, and certificate of analysis.

In addition, if Billy is having a problem with his item, his customized web page can link him to the technical service department's information base where any recorded problems specifically related to that item are displayed. If he cannot find a solution there, he can be routed to a page that gives him a unique priority code that enables him to contact a knowledgable person directly. When Billy receives his priority code, his computer sends his information as well as the item's information directly to the company's Technical Service Department. When Billy calls, the Technical Service Department instantly has his information available, and even the content he was searching for, cutting down on a tremendous amount of paper work. Simplifying this process of obtaining information automatically and quickly ensures customer satisfaction.

### EXAMPLE 2

### RFIDs Associated with E-Gels Used for Separation of Nucleic Acid Molecules

Provided in this example, is an illustrative embodiment of a gel with an associated RFID tag, and the use thereof during and after an electrophoretic separation.

An E-Gel 96 gel (Invitrogen, Carlsbad, Calif.) comprising 1% agarose and SYBR Safe nucleic acid stain (Invitrogen, Carlsbad, Calif.) is provided within an enclosed cassette that also includes the anode and cathode for gel electrophoresis. The outer surface of the E-Gel cassette has an attached writable RFID tag that includes the “locked” information that the gel concentration is 1%. The user, after removing the E-Gel 96 from a foil packet, uses an RFID writer to enter identifier information on the samples run on the gel (the origin of the sample, the sample number or code, and the gel lane the sample is to be loaded in) and to select one of four possible run preferences: 1) long separation, 2) intermediate separation, 3) short separation, and 4) user control. The user enters 1) long separation to the RFID tag using the reader. The user then places the E-Gel 96 onto the E-base (Invitrogen, Carlsbad, Calif.) support/electrical contact unit and connects the E-base to electrical leads that connect to a power supply. The E-base has an integrated RFID tag reader that reads the encoded operating instructions on the RFID tag that is on the cassette. The RFID tag reader transmits the encoded operating instructions to a processing unit that is integrated with the power supply and directs the power supply to maintain a particular voltage across the electrodes of the cassette for ten, twenty, or forty minutes, depending on the sample.

When the electrophoresis run is completed, the gel is placed on a Safe Imager illuminator (Invitrogen, Carlsbad, Calif.) and scanned using a scanner that also includes an RFID reader. The RFID reader receives information from the tag on the cassette that includes information on the gel type (1% agarose), the run conditions, and the source and identity of the samples run on the gel. This information is transferred to a central processing unit that also receives the images scanned by the scanner. The information from the radio identifier frequency tag is integrated with the digital image of the gel and stored in the memory of the processing unit. Software included in the central processing unit can direct comparison of stained bands in the gel lanes and correlate the presence, absence, and relative or absolute intensities of bands with samples.

### EXAMPLE 3

### RFIDs Associated with Gels Used for Separation of Proteins

Provided herein, is an illustrative example of a gel electrophoresis system that includes RFID technology.

A gel electrophoresis/RFID integrated system includes an RFID tagged gel and gel cassette, an RFID reader/writer, an electrophoresis power supply, and an electroblotting apparatus.

A Nu-PAGE gel comprising 4-12% acrylamide and Bis-Tris buffer has a first RFID tag embedded in the gel matrix. The gel is provided in a cassette that has a second RFID tag embedded within the plastic cassette front wall. The cassette-embedded tag includes information on the type of gel enclosed within the cassette (percentage acrylamide and buffer that make up the gel) that is “locked” or non-erasable. The cassette-embedded tag has further memory storage space and is writable, so that sample information can be written to the cassette tag. The gel- embedded RFID tag is also writable. The gel electrophoresis / RFID communicating system further includes an RFID reader/writer such that information on the samples run on the gel can be read from the cassette-embedded tag and written to the gel-embedded tag.

A user scans the Nu-PAGE cassette using an RFID reader that is integrated with a processing unit that displays for the user the gel type (% acrylamide) and buffer to be used with the gel (for example, MES). The user then enters information on the identities of the samples to be run on the gel, and enters the preferred molecular weight range of separation. The integrated unit RFID writer transmits the information to the cassette-embedded RFID tag. The processing unit displays recommended molecular weight markers to use on the gel and directs the power supply to run at 200 volts for 35 minutes. After the gel has run, the user opens the cassette and removes the gel Immediately upon removing the gel, the user uses the RFID reader/writer to read sample information and gel information from the cassette-embedded tag, and transfer it to the gel-embedded tag. The gel is scanned using a scanner that has an integrated RFID reader, and a digitally recorded image is stored in a computer that also receives and stores information from the RFID on the gel type, run conditions, and sample identities.

One half of the gel (comprising a sample set) is electroblotted. Immediately prior to electroblotting, an RFID reader/writer is used to read sample identity information from the gel- embedded tag and transmit the information to a PDVF membrane-attached RFID tag.

The membrane is used for hybridization with an antibody to a DNA binding protein of interest and secondary antibody conjugated to a fluorescent label. The results are also detected by scanning, in which the scanner has an integrated RFID reader that connects to a processing unit that receives and records the image of the filter.

A second half of the gel (comprising a duplicate sample set) is used to divide each sample lane into slices that are subjected to in-gel trypsin proteolysis. The resulting peptides are extracted from the gel slices in tubes that have RFID-embedded tags. An RFID writer is used to transmit sample identity information from the processing unit (which has stored the information relating each gel lane to a sample) to the tube. Additionally, information entered into the processing unit on the slice number (where the slice number related to the molecular weight range of the gel region the slice originated from) is recorded on the slice tube RFID tag.

### EXAMPLE 4

### RFIDs Associated with Arrays for Detection of Expressed Proteins

An array comprising a glass chip having bound single-stranded nucleic acid probes at spatially addressable locations also includes an RFID tag attached at one end of the array. Single-stranded DNA or RNA isolated from a cell type are applied to the array, and after hybridization and washing, a second probe that includes a fluorescent label that hybridizes to a different portion of the target nucleic acid molecule is applied to the array in a sandwich hybridization. After washing, positively hybridizing spots on the array are detected by a fluorescence scanner. The results of the hybridization are recorded by a central processing unit, and a code indicating the hybridization result is written to the RFID tag on the chip using an RFID writer. Chips that have positive hybridization to nucleic acid markers of one type are used for on-chip PCR analysis of relevant genes. The PCR products are transferred to tubes that are RFID tagged with sample identification information. The positive hybridization result is written to the tag using an RFID writer. A code indicating the primer sequences used is also written to the tag on the tube. The PCR product in the tube is directed to an automated sequencer that reads the tag and, on completion of the sequencing run, inputs sequence data to a processing unit that, by reading the RFID tag on the tube, correlates it with, sample and detection and PCR procedure information.

### EXAMPLE 5

### Functional Biochips Used for Sample Processing and Analysis

Biological samples, such as a serum samples, are used to detect the presence of cancer biomarkers using an automated biochip system. The biochip system has multiple biochips, each of which is capable of performing multiple functions in parallel.

A serum sample is applied to a biochip that has multiple depressions, each of which includes a different surface-bound antibody that binds a cell surface marker associated with a particular malignancy. The sample is mixed gently by physical rocking of a platform that supports the chip and intermittent use of acoustic elements built into the surface of the chip. After one hour of incubation, washes are performed by transferring liquid buffer across the chip. An second antibody having an attached fluorescent label is used to detect bound cancer cells using an optical scanner. The positive fluorescence result is written to the chip by a reader that interfaces with a processing unit that also receives data from the scanner. The RFID tagged chip transmits a signal to the reader that the detection result was positive. The system then begins a protocol for nucleic acid detection, in which the chip having bound cells is subjected to cell lysis conditions (heating in hypotonic buffer) and a PCR reaction is performed to detect a splice variant of a gene expressed in cancer cells.

### EXAMPLE 6

### Use of RFID Identifiers in a Proteomics Workflow

A proteomics workflow for the identification of expressed proteins is exemplified in FIGS. 4A and 4B. An RF tag is associated with a cell culture plate, inside of which cells are cultured. A researcher uses an RFID writer, which optionally can be connected to a computer processor, such as a computer processor of a personal computer, to write information to the RFID tag on the culture plate. The technician can write the experiment number, a cell type of cells growing on the plate, growth medium and temperature, a brief description of any special conditions used in culturing cells in gown in the plate, the initials of the technician performing the experiment, and the date the culture was initiated on the RFID tag on the cell culture plate. This information can then be read by an RFID reader on the lab bench of the technician, which can be associated with a computer processor and optionally an RFID writer. A computer display associated with the computer processor can then display the information read from the cell culture plate, and the computer processor can communicate the information received from the reader to a database for storage. The technician can then read information about the cell culture on the display, which can include retrieving the information from the database using the computer processor, and then the technician can use an input function associated with the computer processor, such as a computer keyboard, and enter information into the computer processor that relates to extraction solutions and conditions to be used to extract proteins from the cell culture. Additionally, the computer processor can associate an extraction number with this extraction. The computer then can transmit information to the RFID writer, or information can be entered from the technician directly into an RFID writer, which then writes information about the cell culture, the extraction conditions, and/or descriptive words such as “protein extraction” to an RFID tag on a tube that will be used to hold a protein extraction solution. This process can be repeated for a series of extraction conditions which can be the same or different, and which are used to extract proteins from the same or a different series of cell cultures. As a non-limiting example, two cell types can be analyzed in the experiment described above, a non-transformed cell culture, and a cell culture that is transformed by recombinant expression of an oncogene. The RFID on cell culture plates associated with the two different populations of cells will include information to identify the population of cells inside the cell culture plate.

The series of communications between RFID tags, writers, readers, a computer processor(s), and a computer storage device, such as a hard drive, disclosed in the preceding paragraph, can then be used to transmit, receive and store information for subsequent steps of a protein expression profiling workflow. For example, details regarding a protein purification column and chromatography conditions used to purify proteins that are contained in the protein extract, can be transmitted using an RFID tag associated with the column and/or an RFID tag associated with tube(s) used to collect fractions that have been applied to the column. Then, details identifying samples loaded into particular lanes of a polyacrylamide gel can be written onto an RFID tag associated with the gel, as well as details regarding molecular weight markers that are run on the gel that are stored in a tube provided by a manufacturer, which includes an associated RFID tag. After the gel is run, information regarding the details of the gel run can be written to the RFID tag associated with the gel. Next, after staining proteins separated on the gel, an image scanner used to digitize an image of the gel , or a computer processor associated therewith, can read information from the gel RFID tag and associate this information with the gel image and with other information from the protein expression workflow used to generate protein samples that are loaded on the gel. Furthermore, using information obtained directly or indirectly from the gel RFID tag that was obtained from the RFID tag associated with the molecular weight markers, the computer processor associated with the imager can calculate molecular weights of bands on the scanned gel image. Furthermore, the computer processor can identify bands that are unique to one of the two populations of cells analyzed in the experiment.

Finally, the computer processor can then scan a database of protein molecular weights for proteins from a species of the cells originally included in the cell culture plate from which the workflow initiated, and identify potential proteins that match molecular weights of proteins on the gel that are present in only one of the two populations (i.e. are differentially expressed). The database can be provided by a provider of research reagents whom can then present to the technician, product numbers for antibodies that recognize the proteins that are differentially expressed, and optionally an ordering function for ordering one or more of the antibodies. Furthermore, the technician can search for the antibodies in a laboratory freezer using an RFID reader that is associated with the laboratory freezer, and RFID tags that are associated with vials of antibodies inside the freezer. In an optional confirmatory experiment, the provider of research reagents can identify the specific reagents that can be used by the technician to confirm the results of the initial expression profiling experiment, and/or to extend the findings of the expression profiling experiment. The technician can use an RFID reader associated with the laboratory freezer or other storage area, to determine whether the lab has the necessary reagents and kits to perform the confirmatory experiment and to double check that the reagents and kits are not expired. Finally, during any and/or all steps of the expression profiling experiment, an expiration date associated with a vessel containing a reagent used in any of the reactions in the workflow, can be read by a reader, which can be associated with an instrument, to assure that the reagent is not expired. For example, a power supply used to run a polyacrylamide gel, can check the expiration date including on an RFID identifier associated with the polyacrylamide gel, to assure that the gel is not expired. Thus, during the workflow, the RFID reader, writer, a laboratory computer system, and a computer system of a provider of biological research reagents, which all can be part of a LIMS system, can communicate to improve the success and efficiency of the workflow.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Section headings provided herein are for convenience only, and are not intended to limit the scope of the invention. Although the invention has been described with reference to the above examples, it will be understood that modifications and variations are encompassed within the spirit and scope of the invention. Accordingly, the invention is limited only by the following claims.

1-46. (canceled)

47. A system for analyzing samples using RFID-tagged biological research reagents, comprising:
1) at least one RFID tag reader;
2) a powered device for performing a biological research function on a sample;
3) a processing unit, wherein the processing unit is operatively linked to the RFID tag reader and the powered device for performing the procedure, wherein the processing unit can store information read by the reader, link information read by the reader to information on the parameters or results of the performed function, and/or use information received by the reader to affect an output of the powered device.

48. The system of claim 47, wherein said biological research function is an assay, a detection, an electrophoretic separation, a reaction, a biochemical synthesis, or a sample processing step.

49. The sample analysis system of claim 47, further comprising one or more RFID-tagged vessels, supports, or structures that can hold a sample to be analyzed using the system.

50. The sample analysis system of claim 49, wherein the one or more RFID-tagged vessels, supports, or structures hold or support the sample during at least a portion of the procedure performed on the sample by the powered device.

51-52. (canceled)

53. The sample analysis system of claim 47, wherein the powered device comprises a scanner for fluorescence, radioactive emission, or colorometric detection.

54. (canceled)

55. The sample analysis system of claim 47, wherein the powered device comprises a thermocycler.

56. The sample analysis system of claim 47, wherein the powered device comprises a power supply and circuitry to provide a signal source to electrode configurations, electromagnetic elements, Peltier elements, acoustic elements, or microfluidic devices for cell or biomolecule separation.

57. The sample analysis system of claim 47, wherein the processing unit can direct at least one procedure using information read from an RFID tagged biological reagent using the reader.

58. The sample analysis system of claim 47, wherein the processing unit can integrate and store sample information read from an RFID tagged biological reagent with information on the parameters of a procedure performed by the system.

59-62. (canceled)

63. The sample analysis system of claim 47, wherein the system also has an RFID writer that can enter information on the parameters or results of at least one procedure performed by the system on an RFID tag associated with a biological research product that holds, contains, or supports a sample.

64-78. (canceled)

79. A biological research system, comprising at least two instruments or reagents that are used in a first biological workflow, wherein at least two of the instruments and/or reagents comprise a radio-frequency identifier (RFID) tag, an RFID reader or an RFID writer.

80. The biological research system of claim 79, wherein the workflow is gene or protein expression profiling, RNAi analysis, or protein-protein interaction analysis.

81. The biological research system of claim 79, wherein the workflow comprises a gel or microarray having an RFID tag associated therewith.

82. The biological research system of claim 79, wherein the system is part of a laboratory information management system.

83-97. (canceled)
